(19) United States
(12) Patent Application Publication
(10) Pub. No.: US 2026/0325577 A1
(43) Pub. Date: Oct. 22, 2026

(54) GENETICALLY ENGINEERED BONE MARROW DERIVED MYELOID CELLS FOR TREATMENT OF CENTRAL NERVOUS SYSTEM TUMORS

(71) Applicant: THE RESEARCH INSTITUTE AT NATIONWIDE CHILDREN'S HOSPITAL, Columbus, OH (US)

(72) Inventors: Prajwal RAJAPPA, COLUMBUS, OH (US); Alessandro CANELLA, COLUMBUS, OH (US)

(21) Appl. No.: 19/106,401

(22) PCT Filed: Sep. 6, 2023
(86) PCT No.: PCT/US2023/032123

§ 371 Date: Feb. 25, 2025

Related U.S. Application Data
(60) Provisional application No. 63/403,961, filed on Sep. 6, 2022.

(51) Int. Cl. A61K 35/15; A61K 9/00; A61K 38/20; A61K 40/17; A61K 45/06; A61K 48/00; A61P 35/00; C07K 14/55; C12N 5/0786; C12N 5/0787; C12N 15/867

(52) U.S. Cl. CPC A61K 35/15; A61K 9/0019; A61K 38/2013; A61K 40/17; A61K 45/06; A61K 48/005; A61P 35/00; C07K 14/55; C12N 5/0642; C12N 5/0645; C12N 15/867; A61K 2239/31; A61K 2239/38; A61K 2239/47; C12N 2510/00; C12N 2740/15043

(57) ABSTRACT

A method of treating or preventing central nervous system cancer in a subject in need thereof is described. The method includes administering to the subject a therapeutically effective amount of myeloid cells modified to express interleukin-2. A population of genetically engineered myeloid cells (GEMys) comprising bone marrow derived myeloid cells that have been genetically modified to express interleukin-2 is also described.

### CROSS-REFERENCE TO RELATED APPLICATIONS

This application claims priority to U.S. Provisional Patent Application Ser. No. 63/403,961, filed Sep. 6, 2022, which is incorporated herein by reference.

### BACKGROUND

In the United States, approximately 60,000 new cases of brain tumors have been diagnosed from 2012 to 2016, and nearly 26% were gliomas. Tumors of the central nervous system (CNS) are one of the most prevalent solid tumors and the third leading cause of cancer-related death in adolescents and young adult (AYA) patients (from 15 to 39 years of age). More importantly, there is no established therapeutic protocol for treating gliomas in AYA patients. At the time of diagnosis, two-thirds of AYA patients affected by glioma are histologically low-grade (LGG, WHO grade I and II), and one-third are high-grade gliomas (HGG, WHO grade III and IV). LGG represents a heterogeneous group of primary tumors derived from neuroglial cells (Youssef, G. and J. J. Miller, Curr Neurol Neurosci Rep, 2020. 20(7): p. 21), and in LGG AYA patients the most frequent histological types are astrocytoma (pilocytic grade I, diffuse grade II) and oligodendroglioma (grade II). Papageorgiou, JCO Oncol Pract., 2020, 16(4): p. 155-162. In these patients, the five-year progression free survival (PFS) after total resection is above 90%, but it significantly decreases to 55% in those with residual disease. Wisoff, J. H., et al., Neurosurgery, 2011. 68(6): p. 1548-54; discussion 1554-5. Despite the favorable response of the patients to the standard of care, which consists of total resection followed by chemo-radiotherapy, a significant number of AYA patients experience tumor relapse, associated with a faster progression to HGG and a significant reduction of survival to 1-3 years post-relapse. Burgers, V. W. G., et al., J Natl Compr Canc Netw, 2021. 19(3): p. 240-246.

In the last decade, efforts have started to characterize the tumor biology and genetic heterogeneity of gliomas, but the molecular processes by which tumors relapse, and transform into high-grade disease, remain for the most part, unknown. The glioma microenvironment is a pro-tumoral tridimensional architecture mainly constituted by a network of cancer cells, stromal cells (immune cells, fibroblasts, endothelial cells), blood vessels, and extracellular matrix. The stromal components of the TME support homing and proliferation of the cancer cells through the release of pro- or anti-inflammatory chemokines, exosomes, pro-angiogenetic factors, prosurvival chemokines, and growth factors. Guo, S. and C. X. Deng, Int J Biol Sci, 2018. 14(14): p. 2083-2093. Moreover, the TME assists in the development of immune escape mechanisms that protect the cancer cells during tumor progression by hindering cancer immune surveillance, led by innate and adaptive immune cells. Cancer cells facilitate the immunosuppressive reprogramming of myeloid cells infiltrating the TME (Bejarano et al., Cancer Discov, 2021. 11(4): p. 933-959) through the release of cytokines (IL4, IL10, and TGFβ), restricting the recruitment and activation of cytotoxic effector immune cells, such as cytotoxic T lymphocytes (CD3+ CD8+, CTLs), dendritic (DCs) and natural killer (NK) cells. Kmiecik, J., et al., Journal of Neuroimmunology, 2013.264(1): p. 71-83. Interestingly, glioma tumor progression is characterized by a significantly increased infiltration of myeloid cells and a reduction of infiltrating cytotoxic T and NK cells in the TME. Grabowski et al., J Neurooncol, 2021. 151(1): p. 3-12. In fact, approximately 50% of the immune cells in the TME are myeloid cells, either resident (microglia) or recruited from the bone marrow (bone marrow-derived myeloid cells—BDMCs).

Myeloid cells in the TME can have a range of pro-activating (M1-like) or immunosuppressive (M2-like) phenotypes (Gabrilovich, D. I. and S. Nagaraj, Nat Rev Immunol, 2009. 9(3): p. 162-74), and the balance between M1-like and M2-like myeloid cells, together with the infiltration of immunosuppressive regulatory T cells (Tregs) in the tumor niche, are clinically prognostic. Heimberger et al., Clin Cancer Res, 2008. 14(16): p. 5166-72. To that end, increased tumor infiltration of Tregs and M2-like macrophages have been associated with reduced sensitivity to drug treatments, including immunotherapies, and with a significant reduction of PFS in glioma patients. Lohr et al., Clin Cancer Res, 2011. 17(13): p. 4296-308. In the last decade, immunotherapy was considered a major advance for the treatment of several tumor types, but it has shown very limited efficacy in glioma. This failure is thought to be mainly due to the presence of the blood brain barrier (BBB), which hampers the ability of these drugs to reach the TME, and to immune-escape mechanisms mediated by cancer cells and immunosuppressive myeloid cells. Bao, Z., et al., Front Med, 2021. 15(4): p. 551-561. The reduction of chemo attractant and immune stimulatory molecules within the TME, and the immunosuppression promoted by myeloid and Treg cells, appear to hamper the recruitment and activation of intratumoral cytotoxic immune cells such as CTLs and NK cells. Marvel, D. and D. I. Gabrilovich, J Clin Invest, 2015. 125(9): p. 3356-64. Interleukin-2 (IL-2) is a 15.5 KDa cytokine secreted by activated T lymphocytes and one of the master activators of naïve T cells. Ross, S. H. and D. A. Cantrell, Annu Rev Immunol, 2018. 36: p. 411-433. The interaction between IL2 and its receptor triggers JAK-STAT and Mtor signaling pathways associated with cellular growth, proliferation, and cell cycle progression to drive the cellular differentiation from the naïve to the effector T cell phenotype. On the other hand, excessive or prolonged IL2 stimulation of T lymphocytes can affect the activation status and lead to T cell exhaustion or inactivation. Liu, Y., et al., Nat Immunol, 2021. 22(3): p. 358-369.

Myeloid cells have previously modified to express and release other cytokines, such as interferon-γ, IL-9, IL-15, IL-18, and IL-21, which are involved in the immune activation and recruitment of myeloid and cytotoxic lymphoid cells, to improve the killing of tumor cells and prevent glioma progression. US Patent Publication No. 2022/0127575. However, there remains a need for myeloid cells which have been modified to express IL-2, as well as additional methods for treating central nervous system tumors such as gliomas.

### SUMMARY

Gliomas are the most prevalent type of brain tumors and one of the leading causes of cancer related death in the adolescent and young adult population (AYA). Two-thirds of glioma AYA patients are affected by low-grade gliomas (LGGs), but there are no specific treatments. Therefore, a percentage of LGG patients experience tumor relapse and malignant progression to high-grade glioma which leads to fatal outcomes. In part, malignant progression is potentiated by the immunosuppressive stromal component of the tumor microenvironment (TME) underscored by M2-macrophages and a paucity of cytotoxic T cells. As a result, first-line immunotherapies have failed to improve outcomes for patients with progressive high-grade gliomas.

The efficacy of an in vivo approach that demonstrates the potential for a novel cell-mediated innate immunotherapy designed to abrogate immunosuppressive mechanisms within the glioma TME and enhance the recruitment of activated effector T cells is described herein. A single dose of engineered bone marrow-derived myeloid cells that release Interleukin-2 (GEMys-IL2) was used systemically to treat mice with LGG tumors. The results demonstrate that GEMys-IL2 efficiently crossed the blood brain barrier (BBB), infiltrated the glioma microenvironment, and reprogrammed the infiltrating immune cell composition and transcriptome. In addition, GEMys-IL2 impaired tumor progression and extended survival in a LGG immunocompetent mouse model. The results demonstrated that GEMys-IL2 has a therapeutic effect in vivo, thus supporting its application as a novel immunotherapy.

### BRIEF DESCRIPTIONS OF THE DRAWINGS

The present invention may be more readily understood by reference to the following figures, wherein:

FIGS. 1A-1G provide graphs and images showing the glioma progression from the low- to high-grade affect trafficking and activation of CD3+ T cells in vivo. A)-B) Analysis of the tumor infiltrating immune cells by CyTOF in no tumor (NT), low-grade (LGG), and high-grade glioma (HGG) RCAS/t-va murine model (n=3). A) Representative UMAPS obtained using unsupervised clustering in OMIQ and B) Percentage of each cell type out of total CD45+ cells. C)-F) IPA analysis of the differential gene expression of tumor infiltrating T lymphocytes (TILs) isolated from RCAS/t-va LGG and HGG. The data were generated by scRNAseq (n=3). C) IPA graphical summary of the most important downregulated pathways. D) IPA analysis of the TILs trafficking (p<0.05). E) IPA analysis of the TILs tissue morphology (p<0.05). F) IPA analysis of the dysregulated pathways associated with T cell activation and proliferation. G) Analysis by cytokine array of the TME protein expression of IL2 in brains isolated from RCAS/t-va LGG, and HGG animals (tumoral mass in right hemisphere, n=3-4). Unpaired two-tailed student's t-test calculated statistical significance. *P<0.05,**P<0.01.

FIGS. 2A-2F provide graphs and images showing the generation of bone marrow-derived mature myeloid cells, engineered for the release of IL2 for the treatment of LGGs in vivo, and clone selection. A) Summary of the design of the novel cell-based immunotherapy (BM=Bone Marrow, HPCs=Hematopoietic Progenitor Cells, LV=Lenti Viral particles). B) RT-qPCR analysis of the IL2 expression in vitro in GEMys expressing the empty vector (EV) or -IL2 at day 3 post-lentiviral (LV) transduction. Results are expressed as fold-change of IL2 in GEMys-IL2 as compared with GEMys-EV (n=8). C) Quantification by ELISA of the IL2 secreted in the supernatant of GEMys in culture for 3 days post-LV infection (n=6). D-F) The stable expression of IL2 was reached after one week of clone selection in culture media supplemented with 1 μg/ml of puromycin. To confirm the positive selection of the transduced cells, the GFP positivity was evaluated by D) Immunofluorescence analysis (n=3, images of the green fluorescence have been acquired with 20× of magnification, scale=5 μm), and E) Flow cytometry (n=3). F) RT-qPCR of IL2 gene expression in GEMys-IL2 in selection media. Results are expressed as the fold-change of IL2 expressed in GEMys-IL2 cells, as compared with GEMys-EV (Fc=1, n=5). Unpaired two-tailed student's t-test calculated statistical significance. *P<0.05, **P<0.01, ***P<0.001, ****P<0.0001.

FIGS. 3A-3F provide graphs and images showing the characterization of the myeloid cell composition of GEMys-EV and GEMys-IL2 and the assessment of activation of primary murine CD8+ T cells in vitro. A) CyTOF analysis of the composition of myeloid cells in GEMys-EV and GEMys-IL2 cultivated in selection media (puromycin 1 μg/ml, n=3). B) Evaluation of the average of the percent of myeloid cells in GEMys-EV and IL2 (n=6). C) Primary CD8+ T cells were isolated from spleen of healthy syngeneic mice and co-cultured with GEMys-EV or IL2 (T cells:GEMys ratio 4:1) for 24 h. To analyze the T cells transcriptome, CD8+ cells were isolated by CD8+ magnetic negative selection before RNA extraction and the investigation of genes involved in the T-cell activation (Ki67, IRF4, Ki67, CD69, CD25a), or D) exhaustion (Lag3, Tim-3) was performed by RT-qPCR (n=6). E) CyTOF evaluation of the cytotoxic T cell activation (CD25, 4-1BB, CD107a) of primary CD8+ cells co-cultured in vitro for 48 h with GEMys-EV or GEMys-IL2 (T cells: GEMys ratio 3:1). F) Representation of the main goal of the novel cell-based immunotherapy concept in vivo. Unpaired two-tailed student's t-test calculated statistical significance. *P<0.05, **P<0.01.

FIGS. 4A-4I provide graphs and images showing the treatment of RCAS/t-va LGG animals with a single dose of GEMys-IL2 impact on the transcriptome of tumor infiltrating immune cells. LGG animals were treated systemically by retro-orbital intravenous (IV) injection (80 μl) of GEMys-IL2 or vehicle (PBS). Plasma samples, brain tissues and tumor infiltrating immune cells were analyzed 3-5 days post-treatment A) Representation of the induction of gliomagenesis in RCAS model. B) ELISA quantitation of circulating IL2 in the peripheral blood at day 5 (n=6). C) Immune fluorescence of coronal brain sections of RCAS/t-va LGG animals, for the evaluation of the infiltration of GEMys-GFP+ (Green). Brain sections at day 5 were stained with Hoechst (Blue). Green dots in the control slides were artifacts (n=3). Scale=40 um. D) RT-qPCR for the IL2 (n=5) and IFNγ (n=3) gene expression in immune cells infiltrating the TME. E) RT-qPCR evaluation of LAG3, Tim-3, and FoxP3 in immune cells infiltrating the TME (n=4-6). In D) and E) experiments samples were collected at day 4 post-treatment. F) Dendrogram of the unsupervised hierarchical clustering analysis of total RNA sequencing in intratumoral immune cells (n=3) at day 3. Only the top 100 up and downregulated genes were used to generate the heatmap. G) Summary of the gene enrichment analysis by IPA. H) Volcano plot of the differential gene expression of tumor infiltrating immune cells. In total 16691 genes. In red are the genes significantly upregulated, and in blue are the genes significantly downregulated. Unpaired two-tailed student's t-test calculated statistical significance. *P<0.05, **P<0.01. I) IPA prediction analysis of the top 25 upregulated (red) and 25 downregulated (blue) upstream regulators. The calculation of p-values with IPA is performed by right-tailed Fisher Exact Test.

FIGS. 5A-5F provide graphs showing the treatment of RCAS/t-va LGG animals with a single dose of GEMys-IL2 modify the composition and activation of tumor infiltrating immune cells and improve the survival. LGG animals treated by retro-orbital intravenous (IV) injection (80 μl) with 8×10⁶ GEMys-IL2 or vehicle (PBS). IPA enrichment analysis of signaling pathways and biological functions associated with A) Cytotoxic CD8+ T lymphocytes, B) Cytotoxic Natural killers at day 3 post-treatment. A)-B) p-values were calculated by right-tailed Fisher Exact Test. C) Evaluation of the tumor microenvironment inflammation by cytokine array (n=3). D) Characterization of the tumor infiltrating immune cells CD45+, T, and NK cells (n=3-4) at day 4 post-treatment with GEMys-IL2 by mass cytometry. Unpaired two-tailed student's t test calculated statistical significance. *P<0.05, **P<0.01. E) Kaplan-Meyer evaluation of the survival of RCAS/t-va LGG animals treated at day 28 by a single retro-orbital intravenous (IV) injection (80 μl) of 8×10⁶ of GEMys-IL2, or vehicle (PBS). Log-rank Mantle-Cox test calculated the statistical significance. *** P<0.001. F) Weekly evaluation of the tumor burden by IVIS (day 0 was the day prior to treatment). Left panel: Average of radiance of the photon's emission from every animal (n=7). Right Panel: box and whiskers representation of the average of radiance of the experimental groups.

FIGS. 6A-6H provide graphs and images showing analysis of the tumor microenvironment of RCAS/t-va LGG animals treated with a single dose of vehicle or GEMys-IL2. A) IXMC-based molecular imaging analysis of GFP+ cells quantification in brain sections of LGG animals at day 5 post-engraftment with vehicle (PBS) or GEMys-GFP (n=3). Dots represent the GFP intensity colocalized with nuclear staining in 78 (PBS) and 443 (GEMys-GFP) microscopic fields. B) Quantification of TME infiltration by CD45+GFP+ cells by flow cytometry (n=4). C) Gene expression analysis by RT-qPCR of pro-inflammatory genes and associated with cytotoxic T cells activation in tumor infiltrating immune cells. D) Characterization of the tumor infiltrating regulatory T lymphocytes (Tregs, CD45+ CD25hiFoxP3+, n=4) at day 4 post-treatment with GEMys-IL2 by mass cytometry. E) Cytokine analysis of the total protein extract from the TME of LGG animals treated with vehicle (PBS, upper panel), or GEMys-IL2 (lower panel) (n=4). F) ELISA quantitation of IL2 and IFNγ in the TME of LGG 4 days post-treatment (n=4). G) ELISA quantitation of circulating IL2 in the peripheral blood at the endpoints (n=6). H) H&E staining of 5 μm paraffin embedded brain sections of animals at the endpoints (n=6). Unpaired two-tailed student's t-test calculated statistical significance. *P<0.05, **P<0.01,

FIGS. 7A-7E provide graphs showing the tmpact of the treatment with a single dose of GEMys-IL2 on the transcriptome of tumor infiltrating immune cells in vivo. IPA analysis of differential expressed genes in TME immune cells at day 3 post-treatment with Vehicle, or with 8×106GEMys-IL2 (80 μl, n=3). Bar graphs represent the gene enrichment analysis related to signaling pathways and biological functions posttreatment of A) Macrophages, B) Granulocytes, C) Monocytes, D) T helper (CD4+) and E) Dendritic cells (DCs). p-values were determined by right-tailed Fisher Exact Test.

### DETAILED DESCRIPTION

The present invention provides a method of treating or preventing central nervous system cancer in a subject in need thereof is described. The method includes administering to the subject a therapeutically effective amount of myeloid cells modified to express interleukin-2 (IL-2). A population of genetically engineered myeloid cells (GEMys) comprising bone marrow derived myeloid cells that have been genetically modified to express IL-2 is also provided.

### DEFINITIONS

For clarification in understanding and ease in reference a list of terms used throughout the brief description section and the remainder of the application has been compiled here. Some of the terms are well known throughout the field and are defined here for clarity, while some of the terms are unique to this application and therefore have to be defined for proper understanding of the application.

“A” or “an” means herein one or more than one; at least one. Where the plural form is used herein, it generally includes the singular.

Also herein, the recitations of numerical ranges by endpoints include all numbers subsumed within that range (e.g., 1 to 5 includes 1, 1.5, 2, 2.75, 3, 3.80, 4, 5, etc.).

As used herein, the term “subject” can refer to any warm-blooded organism including, but not limited to, human beings, rats, mice, dogs, goats, sheep, horses, monkeys, apes, pigs, rabbits, cattle, etc. When the term is used in the context of a subject needing or requiring compositions of the present application, the term may be referred to as “a subject in need thereof” and include subjects that have been clinically diagnosed (e.g., by a medical professional, e.g., a physician) as being in need of compositions of the present application, subjects that are suspected of being in need of compositions of the present application, subjects at risk for a disease or condition and who may benefit from compositions of the present application, and subjects that are already suffering from a disease or condition and who may benefit from compositions of the present application.

The term “pharmaceutically acceptable,” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “therapeutically effective amount” is intended to qualify the number or amount of an agent which will achieve the goal of decreasing disease severity while avoiding adverse side effects such as those typically associated with alternative therapies. A therapeutically effective amount may be administered in one or more doses. Treatments that are therapeutically effective within the meaning of the term as used herein, include treatments that improve a subject's quality of life even if they do not improve the disease outcome per se.

An “effective amount” generally means an amount which provides the desired local or systemic effect, e.g., effective to stimulate interleukin release, including achieving the specific desired effects described in this application. For example, an effective amount is an amount sufficient to effectuate a beneficial or desired clinical result.

“Treat”, “treating”, and “treatment”, etc., as used herein, refer to any action providing a benefit to a subject at risk for or afflicted with a condition or disease such as cancer, including improvement in the condition through lessening or suppression of at least one symptom, delay in progression of the disease, prevention or delay in the onset of the disease, etc.

“Preventing,” as used herein, refers to any action that decreases the risk that a subject will develop cancer, or that will decrease the risk of progression from a low-grade cancer to a high-grade cancer. Preventing infection can be done in subjects who have an increased risk of developing cancer. Subjects can have an increased risk of developing cancer (e.g., central nervous system cancer) as a result of, for example, radiation exposure, genetic disorder, a family history of CNS tumors, immunodeficiency, stress and a history of previous cancers.

As used herein, the term “cytokine” refers to a small protein (~5-20 kDa) that is important in cell signaling, and in particular immunomodulation. Examples of cytokines include chemokines, interferons, interleukins, lymphokines, and tumour necrosis factors.

### Treatment Using Myeloid Cells Modified to Express Interleukin-2

One aspect of the invention provides a method of treating or preventing central nervous system cancer in a subject in need thereof. The method includes administering to the subject a therapeutically effective amount of myeloid cells modified to express interleukin-2 (IL-2). Myeloid cells can be modified to express increased cytokine (e.g., IL-2) levels by various means, such as cell culturing, exposure to infection, exposure to other cytokines, exposure to drugs known to stimulate cytokine release (e.g., various monoclonal antibodies), or by using genetic engineering to increase cytokine (e.g., IL-2) release. In some embodiments, the modified myeloid cells are genetically engineered myeloid cells (GEMys) that have been genetically modifed to include an IL-2 gene and in particular an exogenous IL-2 gene. In further embodiments, the myeloid cells are modified to express increased levels of IL-2 relative to their baseline levels of IL-2 release.

The modified myeloid cells can be used to treat or prevent central nervous system cancer in a subject. Cancer, as defined herein, is a disease based on the development of cells that contain genetic damage resulting in the relatively unrestrained growth of the cells. The genetic damage present in a cancer cell is maintained as a heritable trait in subsequent generations of the cancer cell line. Cancer is generally named based on its tissue of origin. Cancer which has metastasized will typically retain traits associated with its tissue of origin.

Central nervous system (CNS) cancer is cancer that occurs in a part of the central nervous system, which is made up of the spinal cord and the brain. Parts of the central nervous system include the cerebrum, the cerebellum, the brain stem, the meninges, and the spinal cord. CNS cancers include medulloblastoma, glioma, astrocytoma, oligodendroglioma, germ cell tumor, and ependymoma. In some embodiments, the CNS cancer is a glioma.

A glioma is a type of tumor that starts in the glial cells of the brain or the spine. Symptoms of a glioma vary depending on the specific location. Symptoms of brain gliomas include headaches, vomiting, seizures, and cranial nerve disorders as a result of increased intracranial pressure, whereas a glioma of the optic nerve can cause visual loss while spinal cord gliomas can cause pain, weakness, or numbness in the extremities.

In some embodiments, the subject has been diagnosed as having CNS cancer. A variety of methods can be used to diagnose a CNS tumor or cancer, including medical history, blood tests, urine tests, medical imaging (e.g., MRI or CT scan), X-ray, and biopsy. Biopsy is the preferred method for diagnosing CNS cancer and can be carried out using sterotactic surgery to avoid risk of damage to the brain.

Gliomas are classificed by the type of cell, by grade, and by location. The grade of a glioma is determined by pathological evaluation of the tumor. Grades of gliomas include biologically benign gliomas, low-grade gliomas, and high-grade gliomas (i.e., glioblastomas). Low-grade gliomas are well-differentiated (i.e., not anaplastic) and tend to exhibit benign tendencies and are associated with a better prognosis for the patient. High-grade gliomas are undifferentiated or anaplastic, are malignant, and carry a worse prognosis. In some embodiments, the subject has been diagnosed as having a low-grade glioma. In further embodiments, treatment prevents progression of the tumor from low-grade to high-grade glioma. In yet further embodiments, the method delays malignant progression from low-grade to high grade glioma. Because low-grade glioma is relatively benign, preventing malignant progression to high-grade glioma can significantly improve the prognosis of a patient having glioma. IL-2 is known to be downregulated in low-grade glioma.

Treatment of cancer comprises, but is not limited to, destroying tumor cells, reducing tumor burden, inhibiting tumor growth, reducing the size of the primary tumor, reducing the number of metastatic lesions, increasing survival of the individual, delaying, inhibiting, arresting or preventing the onset or development of metastatic cancer (such as by delaying, inhibiting, arresting or preventing the onset of development of tumor migration and/or tumor invasion of tissues outside of primary cancer and/or other processes associated with metastatic progression of cancer), delaying or arresting primary cancer progression, improving immune responses against the tumor, improving long term memory immune responses against the tumor antigens, and/or improving the general health of the patient with illness. It will be appreciated that tumor cell death can occur without a substantial decrease in tumor size due to, for instance, the presence of supporting cells, vascularization, fibrous matrices, etc. Accordingly, while reduction in tumor size is preferred, it is not required in the treatment of cancer.

Prevention of CNS cancer can be provided for subjects who have an increased risk of developing CNS cancer (e.g., glioma). A subject may have an increased risk of developing CNS cancer if they have one or more risk factors for CNS cancer. Examples of risk factors include radiation exposure, a family history of brain tumors, including genetic disorders such as neurofibromatosis types 1 and 2, tuberous clerosis, Von Hippel-Lindau syndrome, Li-Fraumeni syndrome, Turcot syndrome, Gorlin syndrome, and Cowden syndrome, having a weakened immune system, exposure to environmental toxins such as vinyl chloride, or possible high levels of cell phone use. For prevention of CNS cancer, the modified myeloid cells (e.g., GEMys cells) are preferably administered periodically to the subject.

The effectiveness of cancer treatment may be measured by evaluating a reduction in tumor load or decrease in tumor growth in a subject in response to the administration of the modified myeloid cells. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

In some embodiments, cancer treatment or prevention using modified myeloid cells (e.g., GEMys cells) is combined with another form of cancer treatment suitable for treating central nervous system cancer. Additional methods of cancer treatment include radiation therapy, chemotherapy, targeted drug therapy (e.g., bevacizumab), radiofrequency ablation, cryoablation, thermal ablation, electroporation, alcohol ablation, high intensity focused ultrasound, photodynamic therapy, hormone-blocking therapy, oncolytic virus treatment, chimeric antigen receptor (CAR) T-Cell therapy, administration of monoclonal antibodies, and administration of immunotoxins.

### GEMys Cell Populations

Another aspect of the invention provides a population of genetically engineered myeloid cells (GEMys) comprising bone marrow derived myeloid cells that have been genetically modified to express interleukin-2 (IL-2). More specifically, the myeloid cells have been genetically modifed to include an IL-2 gene and in particular an exogenous IL-2 gene, as well as a suitable expression control sequence. A population of cells refers to a plurality of cells. In some embodiments, the population of cells includes a number of cells sufficient to provide a therapeutically effective dose for treating or preventing central nervous system cancer. In further embodiments, the population of cells includes a plurality of different types of myeloid cells.

Granulocytes, monocytes, macrophages, and dendritic cells represent a subgroup of leukocytes, collectively called myeloid cells. Prinz et al., Nat Immunol., 22; 18(4):385-392 (2017). Myeloid cells are initially formed in the embryo, but various embodiments of the invention are directed to the use of myloid cells are bone marrow-derived. In some embodiments, the myeloid cells comprise macrophages, granulocytes, monocytes, neutrophils, basophils, eosinophils, mast cells, and myloid precursors. In some embodiments, the GEMys cell population can also include a small amount of lymphoid cells, such as T lymphocytes, B lymphocytes, and natural killer cells. See Table 1, provided herein.

In some embodiments, the myeloid cells comprise monocytes, eosinophils, mast cells, and myloid precursor cells. In further embodiments, at least 10%, at least 15%, at least 20%, or at least 25% of these cells (independently for each cell type) are present in the GEMys cell population. The percentages are relative to total cell numbers present in the population.

The myeloid cells (e.g., GEMys cells) can be allogenic or autologous cells. In some embodiments the myeloid cell (e.g., GEMys cell) is a mammalian myeloid cell. Examples of “mammalian” or “mammals” include primates (e.g., human), canines, felines, rodents, porcine, ruminants, and the like. Specific examples include humans, dogs, cats, horses, cows, sheep, goats, rabbits, guinea pigs, rats and mice. Myeloid cells can be obtained from bone marrow. For example, the myeloid cells can be isolated using magnetic negative selection followed by cultivation in media.

Genetic engineering is the modification and manipulation of an organism's genes using technology. New DNA is obtained by either isolating and copying the genetic material of interest using recombinant DNA methods or by artificially synthesising the DNA. A construct is usually created and used to insert this DNA into the host organism. In the case of the present invention, myeloid cells are genetically modified to express IL-2.

A nucleic acid encoding the IL-2 can be cloned or amplified by in vitro methods, such as the polymerase chain reaction (PCR), the ligase chain reaction (LCR), the transcription-based amplification system (TAS), the self-sustained sequence replication system (3SR) and the QP replicase amplification system (QB). For example, a polynucleotide encoding the polypeptide can be isolated by polymerase chain reaction of cDNA using primers based on the DNA sequence of the molecule. A wide variety of cloning and in vitro amplification methodologies are well known to persons skilled in the art.

The IL-2 gene can be operatively linked with an expression control sequence that has a regulatory element such as a promoter (constitutive or regulatable) to drive transgene expression and a polyadenylation sequence downstream of the nucleic acid. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. Suitable promoters include, but are not limited to, a hVMD2 promoter, an SV40 early promoter, RSV promoter, adenovirus major late promoter, human CMV immediate early I promoter, poxvirus promoter, 30K promoter, 13 promoter, sE/L promoter, 7.5K promoter, 40K promoter, C1 promoter, and EF-1α promoter.

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used cytomegalovirus (CMV) promoter) also comprise enhancer sequences. Enhancers can be located upstream, within, or downstream of coding sequences. For example, the nucleic acid encoding the polypeptide can be operably linked to a CMV enhancer/chicken R-actin promoter (also referred to as a “CAG promoter”).

Additionally, the vector can comprise a reporter to identify the transfection/transduction efficiency of the vector. Exemplary reporters include, but are not limited to, EGFR and CD90.1.

Genetic modification of myeloid cells can be carried out using an appropriate vector (e.g., a viral vector) to insert the interleukin-2 gene into the myeloid cells. Examples of suitable vectors include plasmids (e.g., DNA plasmids), bacterial vectors (e.g., a Listeria or Salmonella vector), yeast vectors, and viral vectors. In one embodiment, the vector is a viral vector, such as retrovirus, poxvirus (e.g., an orthopox (e.g., vaccinia, modified vaccinia Ankara (MVA), Wyeth, NYVAC, TROYVAC, Dry-Vax, or POXVAC-TC), avipox (e.g., fowlpox, pigeonpox, or canarypox, such as ALVAC), raccoon pox, rabbit pox, capripox (e.g., goat pox or sheep pox), leporipox, or suipox (e.g., swinepox), adenovirus, adeno-associated virus, herpes virus, polio virus, alphavirus, baculorvirus, and Sindbis virus. In a specific embodiment, the vector is a lentiviral vector.

Retroviral vectors, including lentiviral vectors, are suitable delivery vehicles for the stable introduction of a variety of genes of interest into the genomic DNA of a broad range of target cells. Without being bound by theory, the ability of retroviral vectors to deliver unrearranged, single copy transgenes into cells makes retroviral vectors well suited for transferring genes into cells. Further, retroviruses enter host cells by the binding of retroviral envelope glycoproteins to specific cell surface receptors on the host cells. Consequently, pseudotyped retroviral vectors in which the encoded native envelope protein is replaced by a heterologous envelope protein that has a different cellular specificity than the native envelope protein (e.g., binds to a different cell-surface receptor as compared to the native envelope protein) also can be used.

There are many retroviruses and examples include: murine leukemia virus (MLV), lentivirus such as human immunodeficiency virus (HIV), equine infectious anaemia virus (EIAV), mouse mammary tumor virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). Other retroviruses suitable for use include, but are not limited to, Avian Leukosis Virus, Bovine Leukemia Virus, and Mink-Cell Focus-Inducing Virus. The core sequence of the retroviral vectors can be derived from a wide variety of retroviruses, including for example, B, C, and D type retroviruses, as well as spumaviruses and lentiviruses. An example of a retrovirus suitable for use in the compositions and methods disclosed herein, includes, but is not limited to, lentivirus.

One lentivirus is a human immunodeficiency virus (HIV), for example, type 1 or 2 (i.e., HIV-1 or HIV-2). Other lentivirus vectors include sheep Visna/maedi virus, feline immunodeficiency virus (FIV), bovine lentivirus, simian immunodeficiency virus (SIV), an equine infectious anemia virus (EIAV), and a caprine arthritis-encephalitis virus (CAEV). Customized vectors are commercially available. See for example VectorBuilder®. Lentivirus vectors suitable for insertion of the IL-2 gene are also commercially available. For example, the Lenti-Pac™ lentiviral packaging kit can be used.

Interleukin-2 (IL-2) is a cytokine signaling molecule in the immune system. IL-2 has essential roles in key functions of the immune system, tolerance and immunity, primarily via its direct effects on T cells. IL-2 promotes the differentiation of immature T cells, and increases the cell killing activity of both natural killer cells and cytotoxic T cells. A variety of amino acid sequences for IL-2 are known. The human amino acid sequence for interleukin-2 is provided by NCBI Reference Sequence NP_000577.2.

### Dosage and Administration

The GEMys cells should be administered and dosed in accordance with good medical practice, taking into account the site and method of administration, scheduling of administration, patient age, sex, body weight, the nature and severity of the disorder to be treated or prevented, and other factors known to medical practitioners. As used herein, the term “administer” refers to the placement of a composition into a subject by a method or route which results in at least partial localization of the composition at a desired site such that desired effect is produced. A GEMys cell population, as described herein, can be administered by any appropriate route known in the art including, but not limited to, oral or parenteral routes, including intravenous, intramuscular, subcutaneous, and transdermal administration.

Typically said dose is about 10×10⁶ cells/kg of subject weight or lower, is about 9×10⁶ cells/kg or lower, is about 8×10⁶ cells/kg or lower, is about 7×10⁶ cells/kg or lower, is about 6×10⁶ cells/kg or lower, is about 5×10⁶ cells/kg or lower. In an alternative embodiment said dose may be between about 0.25×10⁶ cells/kg to about 5×10⁶ cells/kg; or more preferably about 1×10⁶ cells/kg to about 5×10⁶ cells/kg. Accordingly in further alternative embodiments the dose may be about 0.25×10⁶ cells/kg, 0.5×10⁶ cells/kg, 0.6×10⁶ cells/kg, 0.7×10⁶ cells/kg; 0.8×10⁶ cells/kg; 0.9×10⁶ cells/kg; 1.1×10⁶ cells/kg; 1.2×10⁶ cells/kg; 1.3×10⁶ cells/kg; 1.4×10⁶ cells/kg; 1.5×10⁶ cells/kg; 1.6×10⁶ cells/kg; 1.7×10⁶ cells/kg; 1.8×10⁶ cells/kg; 1.9×10⁶ cells/kg or 2×10⁶ cells/kg. The dose may, in other embodiments, be between 0.1 and 1 million cells/kg; or between 1 and 2 million cells/kg; or between 2 and 3 million cells/kg; or between 3 and 4 million cells/kg; or between 4 and 5 million cells/kg; or between 5 and 6 million cells/kg; or between 6 and 7 million cells/kg; or between 7 and 8 million cells/kg; or between 8 and 9 million cells/kg; or between 9 and 10 million cells/kg.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

GEMys cells can be supplied in the form of a pharmaceutical composition, comprising an isotonic excipient prepared under sufficiently sterile conditions for human administration. The composition can be sterile. The formulation should suit the mode of administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising a population of GEMys cells will be adapted in accordance with the route and device used for administration.

In some embodiments, the GEMys cells are administered together with a pharmaceutically acceptable carrier. Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets.

Preferably, the cells are administered by injection, e.g., intravenously. The pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

The following example is included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE

### Genetically Modified IL-2 Bone Marrow-Derived Myeloid Cells Reprogram the Glioma Immunosuppressive Tumor Microenvironment

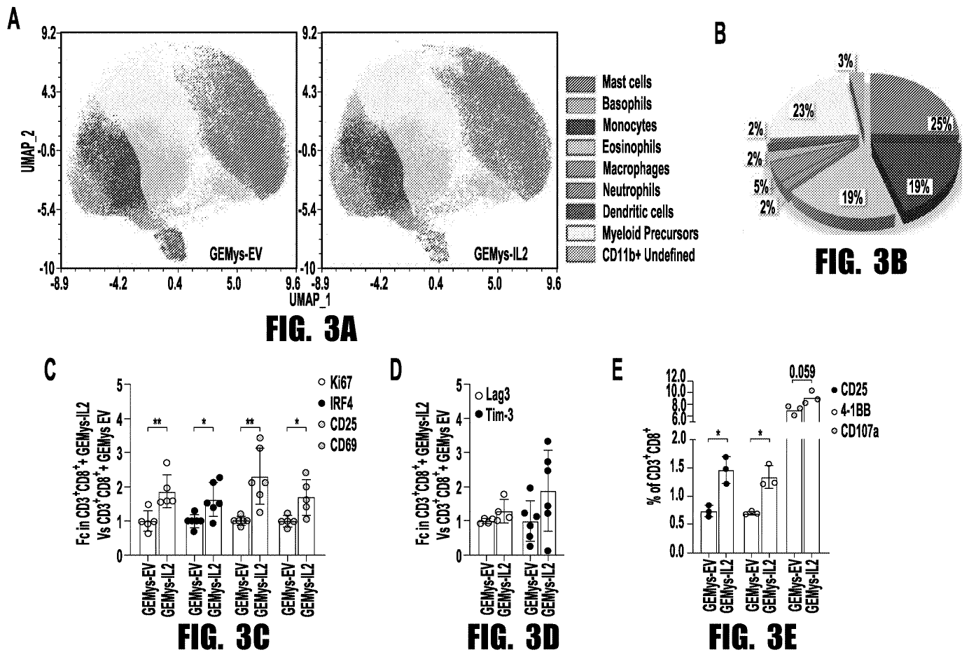

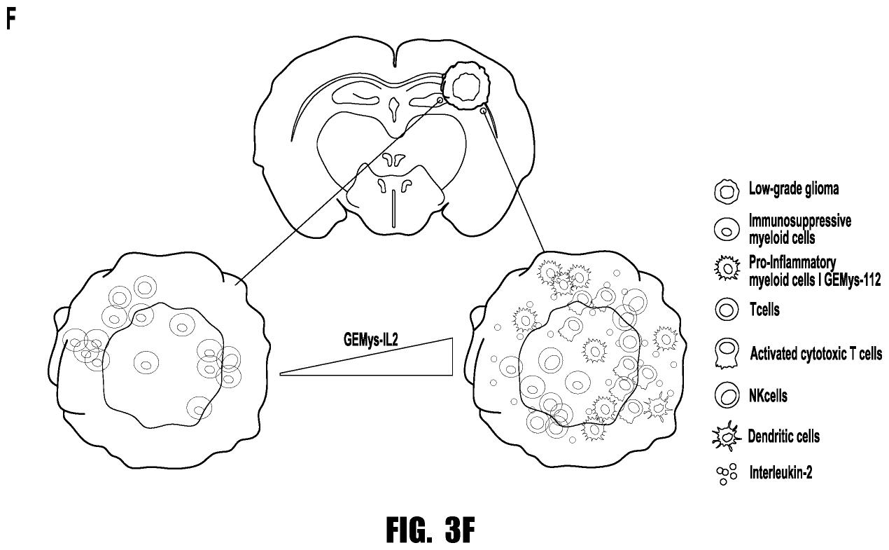

The inventors evaluated a novel therapeutic concept to deliver bone marrow-derived myeloid cells (GEMys) genetically engineered for the stable expression and release of interleukin-2 (IL2) within the TME to promote the recruitment and activation of CTLs against tumor cells (FIG. 3F). They demonstrated that an intravenous injection of a single dose of GEMys-IL2 in LGG mice was sufficient to rapidly recruit and activate more CTLs and NKs in the TME. The GEMys passed the blood brain barrier (BBB) and released IL2 in the glioma microenvironment, thus promoting inflammation, trafficking and activation of cytotoxic T, NK cells, and reprogramming the transcriptome of the immune cells. Importantly, the GEMys-IL2 treatment in vivo delayed tumor progression and significantly improved the overall survival (OS) in LGG bearing animals.

### Results

### Glioma Malignant Progression Effects the Recruitment of T Cells in the TME

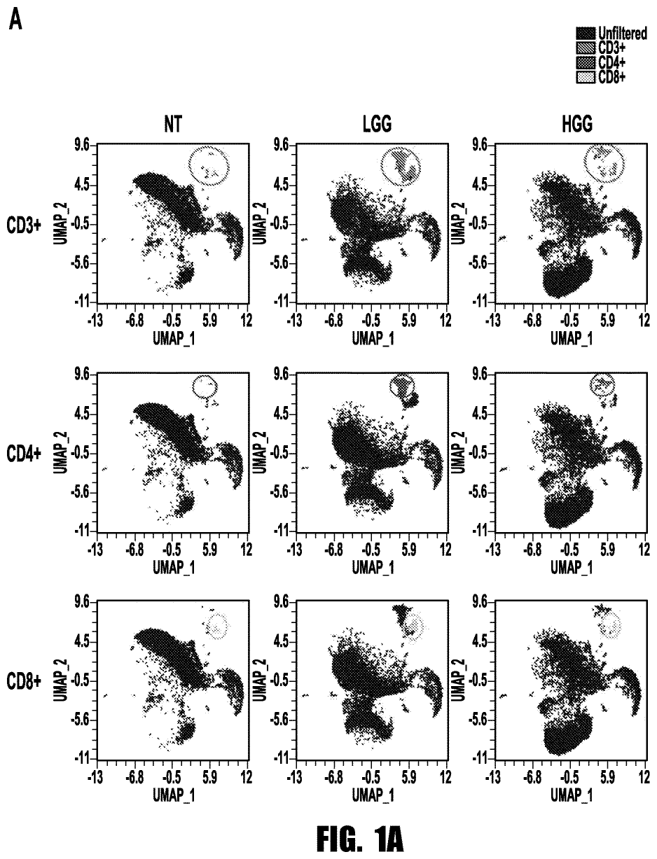

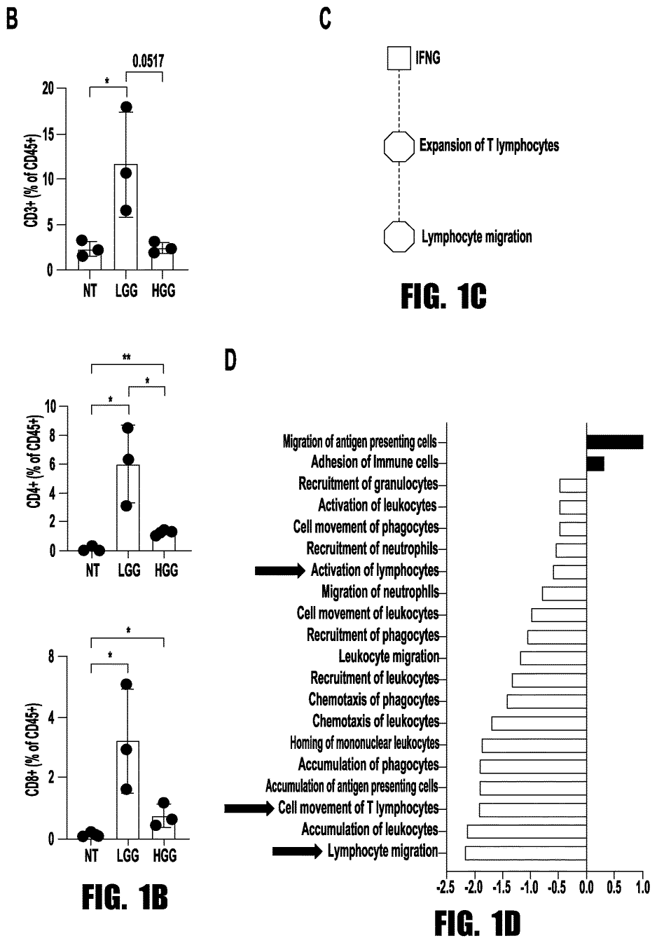

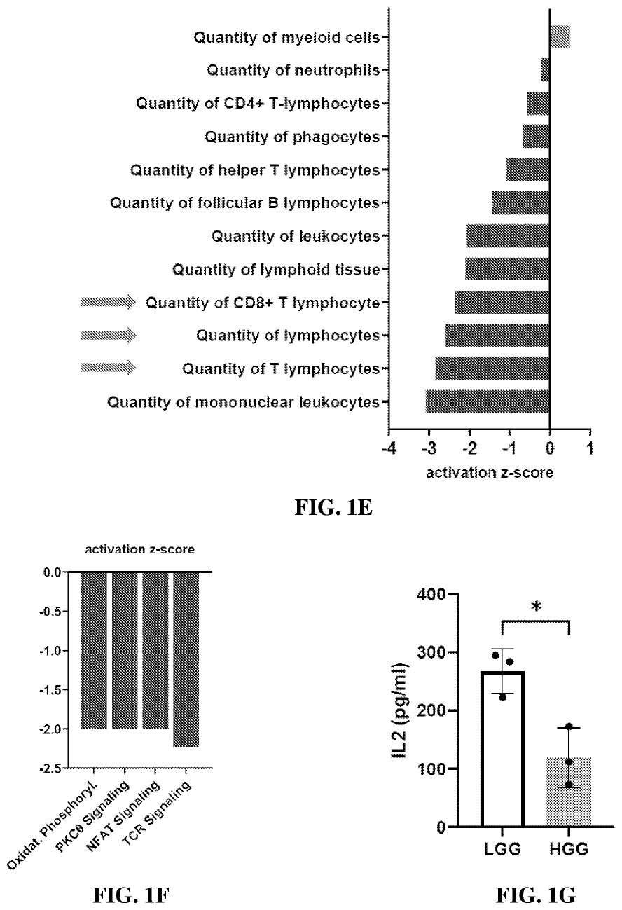

Recent studies have demonstrated that glioma cells reprogram immune cells that are infiltrating the tumor microenvironment to trigger drug resistance, immune-evasion mechanisms, and develop an immunosuppressive phenotype, blocking the recruitment and activation of cytotoxic T and NK cells. Pombo et al., Elife 9. 10.7554/eLife.52176 (2020). In our study, mass cytometry (CyTOF) analysis in brain tissue of RCAS/t-va animals bearing no tumor, LGG, and HGG showed significantly reduced infiltration of CD3, CD4, and CD8 after malignant progression from low- (LGG) to high-grade glioma (HGG) (FIG. 1A-B). Our recent investigation of the tumor infiltrating immune cells during glioma malignant progression revealed four distinct subtypes of myeloid cells with immunosuppressive phenotype, and a striking reduction of tumor infiltrating T, and NK cells after malignant transformation (MT). Rajendran et al., Cell Rep 42(3), 112197 (2023). Moreover, Ingenuity Pathway Analysis (IPA®, Qiagen) of single cell RNA sequencing data (scRNAseq) obtained from the tumor infiltrating lymphocytes (TILs) in HGGs compared with LGGs was used to predict upstream regulators, altered canonical pathways, and disease-related biological functions (GSE221440). As shown in FIG. 1C-F, the analysis highlighted the effect of tumor progression on the immunosuppression of the CD3+ cell compartment (T cells). Indeed, we found a strong downregulation of IFNγ and a reduction of T cell trafficking (FIG. 1C). Furthermore, the gene enrichment analysis by disease and function showed a strong decrease of genes and pathways involved in activation, migration, and trafficking of T lymphocytes (FIG. 1D, red arrows, FIG. 1F) resulting in decreased tumor infiltration by T lymphocytes, particularly by CD8+ T cells (FIG. 1E, red arrows). In addition, signaling pathways associated with T cell activation and proliferation were significantly downregulated (FIG. 1F), such as the T cell receptor (TCR), and the PKCθ signaling pathways. These pathways are critical for effector T cell proliferation, activation and are also associated with naïve T cell differentiation into effector T cells. Courtney et al., Trends Biochem Sci 43, 108-123. 10.1016/j.tibs.2017.11.008 (2018).

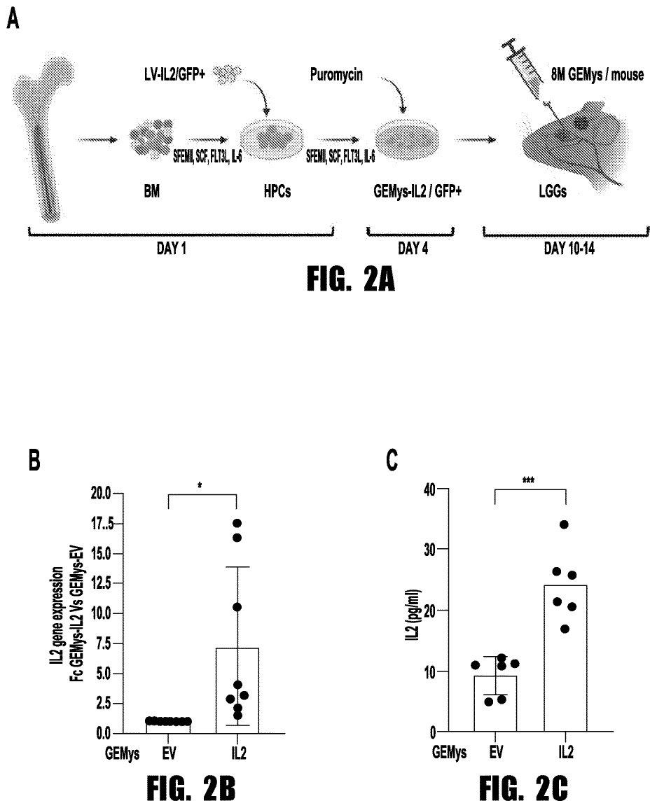

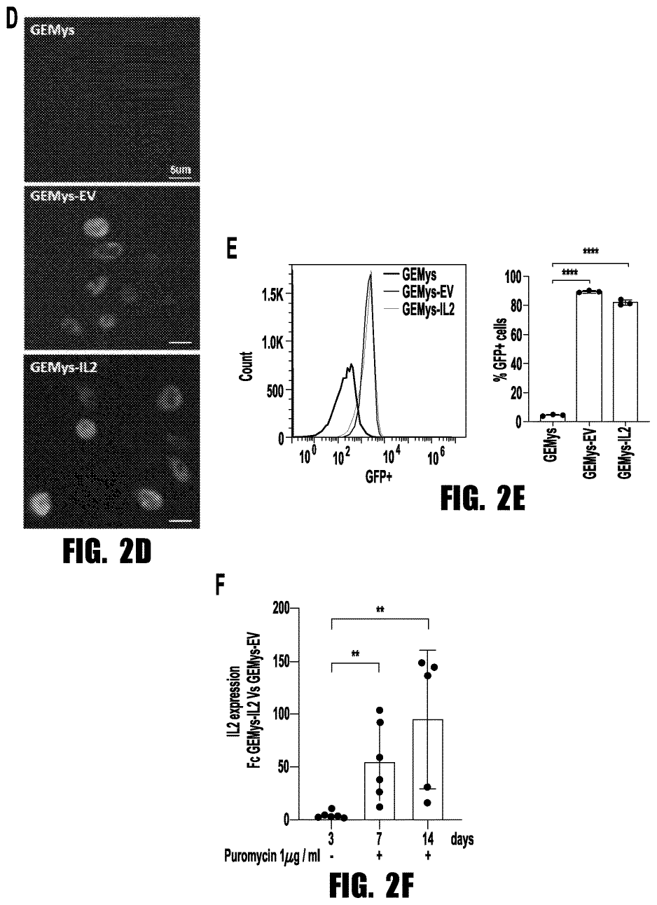

In line with our scRNAseq findings, which revealed a significant reduction of T cell activation in the TME, the cytokine analysis of the glioma microenvironment during malignant progression from low- to high-grade glioma showed a significant downregulation of IL2, CCL21, and CX3CL1 in the tumor milieu, which are associated with T cell recruitment, proliferation and activation (FIG. 1G). Zeng et al., Cancer Res 67, 2331-2338 (2007). Based on these results, we evaluated the ability of engineered bone marrow-derived myeloid cells to reprogram the immunosuppressive myeloid cell-rich TME, to recruit anti-cancer immune cells and to activate CTLs in the glioma microenvironment (FIG. 2A).

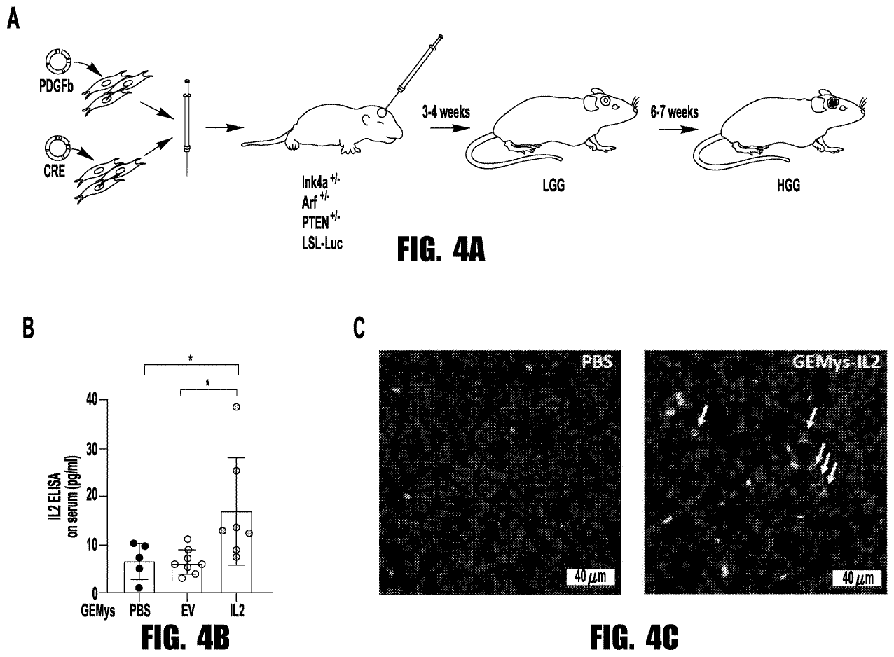

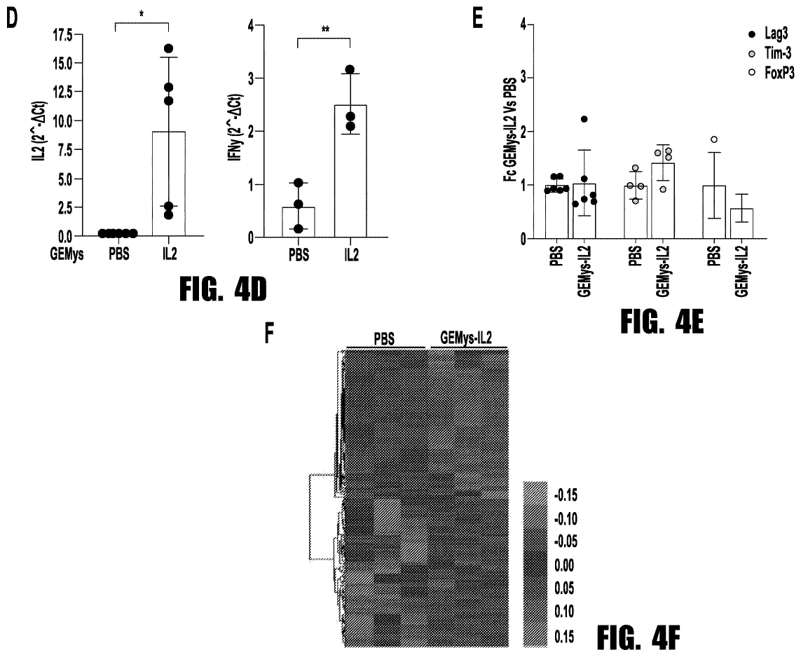

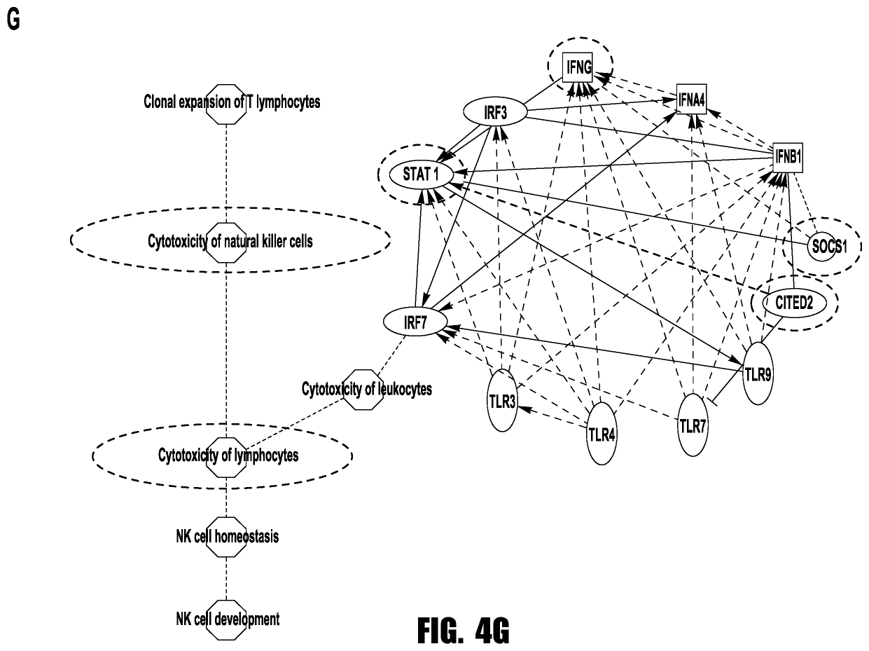

To test our therapeutic approach, we used the RCAS/t-va murine model to recapitulate low to high-grade glioma malignant progression in AYA mice (FIG. 4A), as previously described. Rajendran et al., ibid. In brief, gliomagenesis is triggered by a right hemispheric parenchymal injection of DF1 cells for the release of PDGF RCAS viral particles in pups (day 0-2 of age). This immunocompetent model supports gliomagenesis and tumor progression from low- to high-grade within 8 weeks (FIG. 4A). Kim et al., Cancer Res 72, 6065-6075 (2012).

### GEMys-IL2 Demonstrate Stable Expression of IL2

Three days post-isolation and infection, the GEMys-IL2 population showed a significant upregulation of IL2 gene expression (FIG. 2B) and IL2 secretion in the culture media (FIG. 2C). Unexpectedly, engineered IL2 secreting myeloid cells consistently expressed and secreted more IFNγ than the GEMys-EV cells, and this indirect effect might potentiate the activation of tumor-infiltrating immune cells (FIG. 1A-E). Castro et al., Front Immunol 9, 847 (2018). To select the infected clones, cells were cultivated in selection media for 10 days (GEMys media enriched with puromycin 1p g/ml) prior to the engraftment in mice. To confirm the stable expression of IL2 and the clone selection, GEMys were monitored weekly by fluorescent microscopy, flow cytometry, and quantitative real-time PCR (RT-qPCR) (FIG. 2D-F).

After clone selection, GEMys-EV and GEMys-IL2 myeloid composition were characterized by a large immune CyTOF panel, and the percentage of myeloid precursors were assessed by flow cytometry (Table 1). Samples were gated on CD45+ singlets. Nine similar yet distinct clusters were identified via FlowSOM in both the GEMys-EV and GEMys-IL2 and confirmed by manual gating in OMIQ according to phenotypes reported by Becher B. et al. Nat Immunol 15, 1181-1189 (2014). As shown in FIG. 3A-B and Table 1, we identified Basophils (2.19±0.28%, CD45+ CD11b+ B220− FcRe1a+), Eosinophils (18.87±4.71%, CD45+ CD11b+ SiglecF+), Mast cells (25.13±6.93%, CD45+ CD11b+/− FceR1a+), Neutrophils (4.67±0.90%, CD45+ CD11b+ Ly6G+), Monocytes (19.27±3.25%, CD45+ CD11b+ Ly6G− CD62L+ SiglecF−), Macrophages (1.73±0.26%, CD45+ CD11b+ F4/80+), and Dendritic cells (1.91±2.31%, monocytic: CD45+ CD11b+ CD11c+ MHCII+, conventional: CD45+ CD11b− CD11c+ MHCII+, plasmacytoid: CD45+ CD11b− CD11c+ CD317+), and Myeloid precursors (22.90±4.41%, CD45+ CD11b− FceR1− cKit+). The panel also assessed natural killer cells (0.33±0.39%, CD45+ CD11b− NKp46+), T cells (0.07±0.005%, CD45+ CD11b− CD3+), and B cells (0.002±0.001%, CD45+ CD11b− CD19+), but their numbers were collectively below 0.6% of the composition of both the GEMys-EV and GEMys-IL2 (Table 1). In addition, approximately 2.89% of the CD45+ cells were unidentified myeloid cells (CD45+ CD11b+). These cells expressed CD45 and CD11 b, but none of the distinctive markers used for myeloid characterization (FIG. 3A-B; Table 1). In addition, we did not find any substantial differences in myeloid composition among GEMys-EV and GEMys-IL2. (Table 1).

TABLE 1

| | | GEMys-EV | GEMYS-IL2 |
|---|---|---|---|
| Phenotype | Cell type | % ± SD | % ± SD |
| MYELOID CELLS | | | |
| CD45+ CD11b− CD11c+ MHCII+ (cDC), | Dendritic cells (DCS) | 2.69 ± 3.38 | 1.14 ± 0.24 |
| CD45+ CD11b+ CD11c+ MHCII+ (mDC), | | | |
| CD45+ CD11b− CD11c+ CD317+ (pDC) | | | |
| CD45+ CD11b+ F4/80+ | Macrophages | 1.69 ± 0.26 | 1.78 ± 0.31 |
| CD45+ CD11b+ Ly6G− CD62L+ SiglecF− | Monocytes | 21.00 ± 3.16 | 17.53 ± 2.73 |
| CD45+ CD11b+ B220− FceR1a+ | Basophils | 2.22 ± 0.24 | 2.16 ± 0.37 |
| CD45+ CD11b+ SiglecF+ | Eosinophils | 22.50 ± 3.73 | 15.23 ± 1.42 |
| CD45+ CD11b+ Ly6G+ | Neutrophils | 4.45 ± 1.09 | 4.88 ± 0.83 |
| CD45+ CD11b+/− FceR1a+ | Mast cells | 23.17 ± 8.8127 | 10 ± 5.56 |
| CD45+CD11b−FceR1a−cKit+ | Myeloid Precursors | 20.03 ± 4.82 | 25.77 ± 0.87 |
| CD45+ CD11b+ CD44+ | Undefined CD11b+ | 1.92 ± 0.66 | 3.87 ± 0.81 |
| LYMPHOID CELLS | | | |
| CD45+ CD11b− CD3+ | Lymphocytes T | 0.01 ± 0.01 | 0.01 ± 0.005 |
| CD45+ CD11b− CD19+ | Lymphocytes B | 0.002 ± 0.001 | 0.003 ± 0.001 |
| CD45+ CD11b− NKp46+ | Natural Killer (NK) | 0.45 ± 0.57 | 0.21 ± 0.11 |

### GEMys-IL2 Activate Primary Murine CD8+ T Cells In Vitro

Cytotoxic T lymphocytes lead the adaptive immune anti-cancer response, and their activation is critical to effective immunotherapies. To test the ability of GEMys-IL2 to activate the target cells in vitro, we co-cultured primary murine CTLs with GEMys for 24 h and evaluated the canonical markers associated with cytotoxic T cell activation by RT-qPCR (FIG. 3C). We also evaluated the expression of Lag3 and Tim-3 among the primary markers associated with T cells inactivation/exhaustion in the same experiment (FIG. 3D). Yang et al., Oncotarget 8, 61425-61439 (2017); Avery et al., Proc Natl Acad Sci USA 115, 2455-2460 (2018). The co-culture of murine CTLs with GEMys-IL2 triggered the significant upregulation of Ki67 (1.9-fold), IRF4 (1.6-fold), CD25 (IL2 receptor, 2.3-fold) and CD69 (1.7-fold), as compared with the co-culture of CTLs with control GEMys-EV (FIG. 3C). No significant differences in Lag3 and Tim-3 expression were detected in T cells co-cultured with GEMys-EV or IL2, thus confirming the absence of T cells exhaustion/inactivation in the experimental setting. However, Tim-3 showed a positive but not significant trend in T cells co-cultured with GEMys-IL2 (FIG. 3D).

To further validate the activation of CD3+ CD8+ T cells in vitro, we performed two additional experiments. First, we co-cultured the CTLs with GEMys for 48 hours, and evaluated the expression of cytotoxic T cell activation markers CD25, 4-1BB and CD107a by CyTOF (FIG. 3E). In addition, we also assessed the protein expression of the CD8+ T cell activation marker CD69 by flow cytometry, after 96 hours of incubation with GEMys. CD25, 4-1BB, and CD69 protein expression measured in CD3+ CD8+ T cells co-cultured with GEMys-IL2 (ratio 3:1) were on average respectively 2.0, 1.9 and 4.4-fold significantly higher compared to the levels in T cells treated with control GEMys-EV, thus confirming the results from gene expression (FIG. 3C). Second, we cultured GEMys for three days (80% of confluency) in the absence of puromycin then harvested their media and used it to cultivate CTLs for 48 h. Although the culture conditions were not ideal for the maintenance of primary T cells, the evaluation by RT-qPCR of T cell activation markers showed a significant upregulation of IRF4 (1.4-fold) in T cells cultured in GEMys-IL2 media compared with T cells in GEMys control media (GEMys-EV). Once again, the T cell exhaustion markers Lag3 and Tim-3 were not differentially expressed in CTLs cultivated in media isolated from GEMys-EV or -IL2, suggesting a supportive effect from secreted factors released by GEMys-IL2 on the activation status of CD3+ CD8+ T cells. Of note, GEMys maintained in culture with the same methods for experiments shown in FIG. 3C-D did not show significant differential expression of genes associated with CD8+ T cell activation or exhaustion.

### GEMys-IL2 are Recruited and Infiltrate the Local Glioma Microenvironment

Using a pre-clinical immunocompetent glioma murine model, we tested the efficacy of GEMys-IL2 as a novel cell-mediated immunotherapy for reprogramming the glioma TME prior to malignant progression (FIG. 3F). We engrafted 4-week-old LGG bearing animals with GEMys (FIG. 4A). The RCAS/t-va glioma model also conferred the expression of luciferase in LGG cancer cells during tumor progression. Hambardzumyan et al., Transl Oncol 2, 89-95. 10.1593/tlo.09100 (2009). Therefore, we evaluated the tumor burden at day 25 by measuring the intracranial emission of photons released by the cancer cells using in vivo imaging system (IVIS) to randomize the mice into three treatment groups. Each group was engrafted at day 28 by retro-orbital intravenous injection of PBS (vehicle), 8×10⁶ of GEMys-EV, or 8×10⁶ of GEMys-IL2 cells. Mice were monitored post-treatment daily to evaluate toxicity or stress induced by the treatment and the tumor burden was evaluated by IVIS post-initial GEMys inoculation and at seven days post-treatment. The systemic delivery of syngeneic myeloid cells was well tolerated by the animals, who showed no sign of stress, discomfort, or behavioral issues during the experiments. In addition, no morphological signs of cerebral edema were found in the brain of LGG animals treated with GEMys-IL2 given that historically, cerebral edema was one of the adverse effects documented in patients affected by brain tumors after intratumoral local treatment with recombinant Interleukin-2 (rIL2). In addition, 5 days post-engraftment, peripheral blood samples and brain tissues were isolated for further evaluation. We quantified the amount of IL2 in the serum by ELISA and verified that the amount of circulating IL2 in the animals engrafted with GEMys-IL2 was significantly higher than in animals treated with PBS, or with GEMys-EV (FIG. 4B).

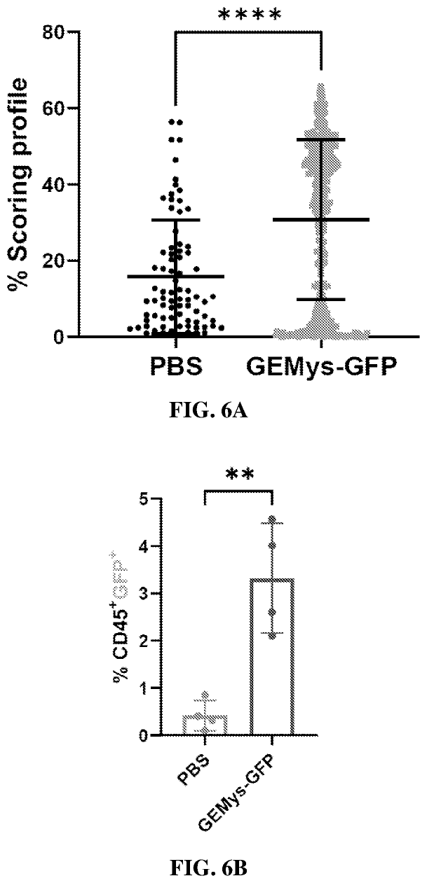

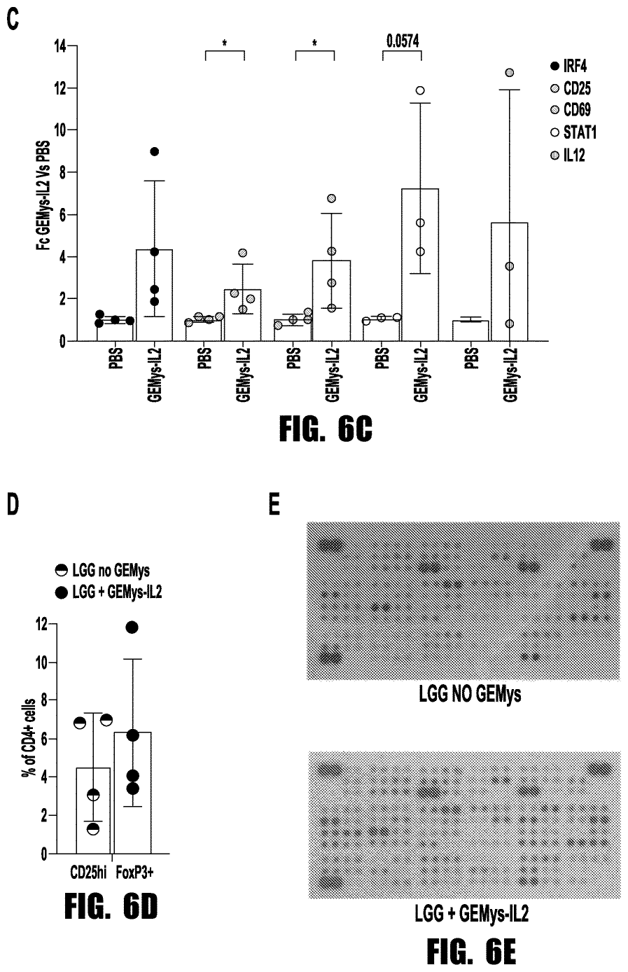

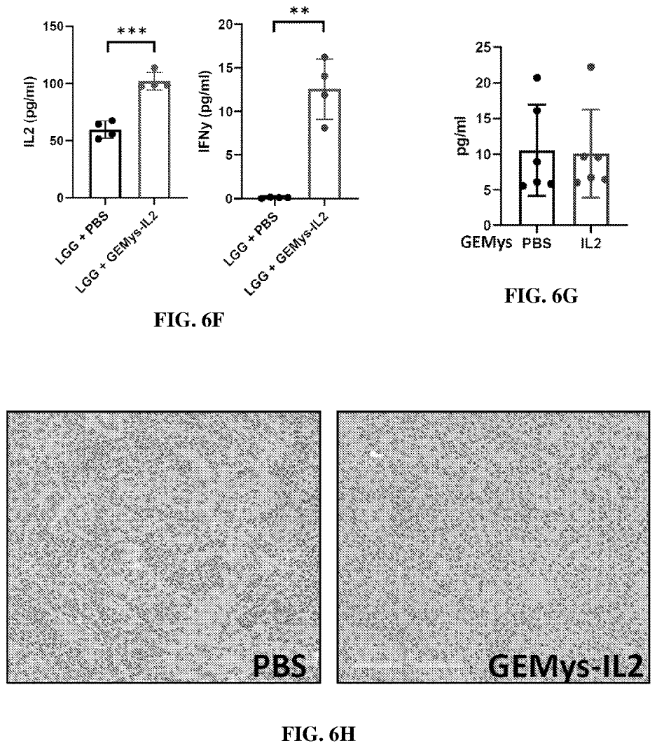

Previously, we demonstrated the recruitment and mobilization of peripheral bone marrow-derived myeloid cells to the glioma TME and therefore we hypothesized that systemically injected GEMys may home to the local glioma microenvironment. Rajappa et al., Clin Cancer Res 23, 3109-3119 (2017). We evaluated by confocal microscopy whether systemically injected GEMys-GFP in LGG animals were capable of passing the BBB and infiltrating the local glioma TME. The analysis of the intratumoral GFP quantification required the application of algorithms to filter out the GFP cellular signal due to artifacts and basal levels of autofluorescence. To that end, we observed GFP-positive myeloid cell infiltration in the glioma microenvironment of in animals engrafted with GEMys-IL2 (FIG. 4C; FIG. 6A,B). These results demonstrate that circulating GEMys pass the blood brain barrier (BBB) and localize to the TME. No GFP-positive cells were found in brain sections from control LGG animals injected with vehicle (PBS, FIG. 4C).

### GEMys-IL2 Regulate Immune Cell Transcriptomes in the Glioma Microenvironment

To assess whether the GEMys-IL2 influence the immune cell transcriptomes, we injected 8 million GEMys-IL2 intravenously. Then, we isolated the immune cells infiltrating the LGG microenvironment on day 5 post-treatment. First, we measured the gene expression of IL2 and IFNγ to evaluate the activation status of the T lymphocytes and myeloid cells. The analysis by quantitative real-time PCR revealed that immune cells isolated from animals engrafted with GEMys-IL2 had a significant increase of IL2 (10-fold) and IFNγ (2.5-fold) expression compared with mice treated with vehicle (FIG. 4D). Remarkably, the treatment of LGG mice with GEMys-IL2 did not induce any significant change in expression of the T cell exhaustion markers LAG3 and Tim-3 (FIG. 4E).

Next, we compared the immune cell transcriptome by total RNA sequencing (RNAseq) in the low-grade glioma microenvironment from animals at day 3 post-engraftment with GEMys-IL2, or with Vehicle (PBS). Unsupervised hierarchical clustering identified 1631 genes that were differentially expressed among the two groups of animals analyzed (1215 genes upregulated and 416 downregulated) (GSE233646, FIG. 4F,H). Of note, we identified the upregulation of genes associated with activation of myeloid (Stat1, CD36), cytotoxic T lymphocytes (IL2ra, CD69, Stat1), and NK cells (Nkg7, IL2rb, CD27) (FIG. 4H). In addition, the Ingenuity Pathway Analysis software (IPA) predicted the increased cytotoxicity and proliferation of T and NK cells, as summarized in FIG. 4G. Furthermore, IPA software analysis of the top 50 upstream regulators, showed that the treatment in vivo of LGG with GEMys-IL2 was associated with the upregulation of IL2 and IFNγ (pro-inflammatory and pro-activators of cytotoxic T cells), but also the upregulation of STAT1 (FIG. 4I, red arrows). STAT1 is critical for the activation and recruitment of CD8+ T cells and for the inhibition of infiltrating suppressive myeloid cells in solid tumors61. The IPA analysis also predicted the downregulation of the master regulators of immunosuppression in M2-like myeloid cells IL10RA, CITED2 and STAT3 (FIG. 4I, blue arrows)62,63. In addition, SOCS1 was predicted to be one of the top downregulated molecules after treatment (FIG. 4G,I). SOCS1 is a specific inhibitor of the JAK/STAT pathway, and regulatory function is performed by specific binding with phosphorylated JAK molecules, followed by the recruitment of E3 ligases, and proteasome degradation. Sharma et al., Front Pharmacol 10, 324 (2019). The gene enrichment analysis also demonstrated the significant upregulation of a network controlling the trafficking of immune cells and of genes involved in the regulation of the inflammatory response. To corroborate the RNA sequencing signature of the tumor infiltrating immune cells, we also assessed the expression of genes in the context of T and myeloid cell activation (FIG. 6C). The analysis by RT-qPCR demonstrated the upregulation of CD25, CD69, STAT1, IRF4, and IL12 in immune cells isolated from the TME of LGG animals engrafted with GEMys-IL2, in comparison to vehicle (PBS). However, only CD25 and CD69 reached statistical significance.

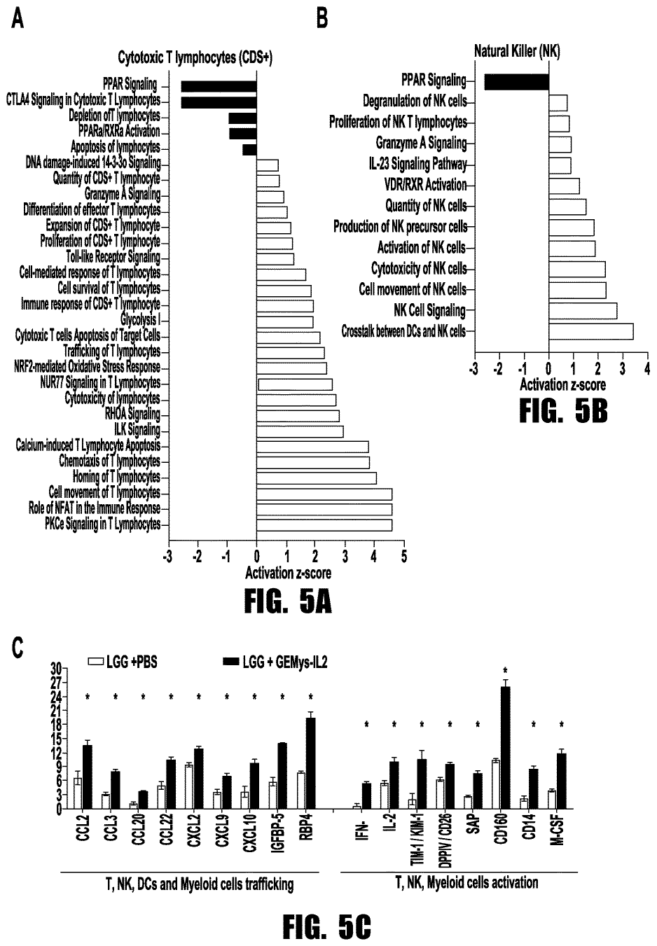

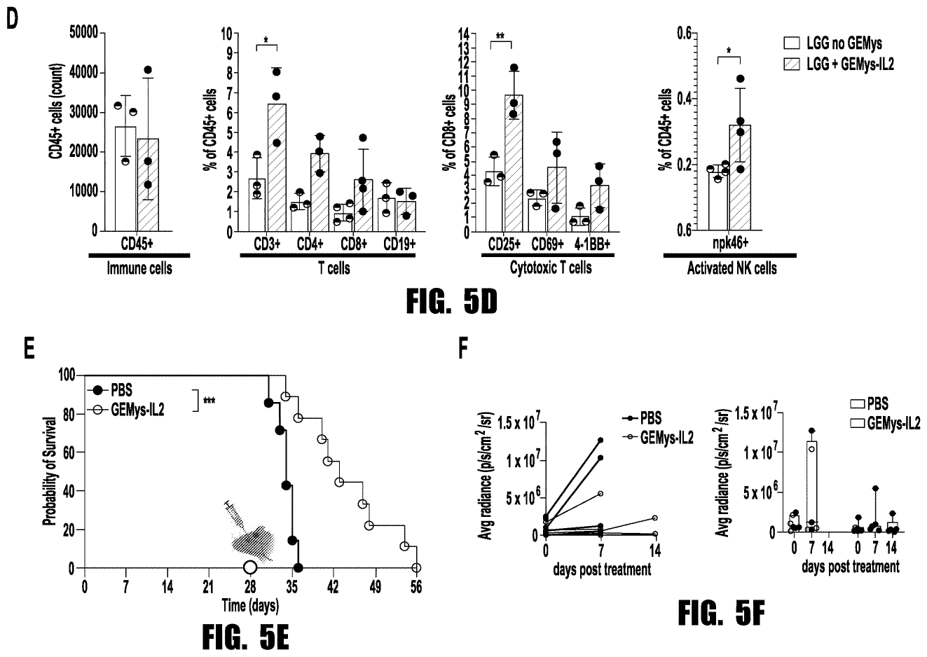

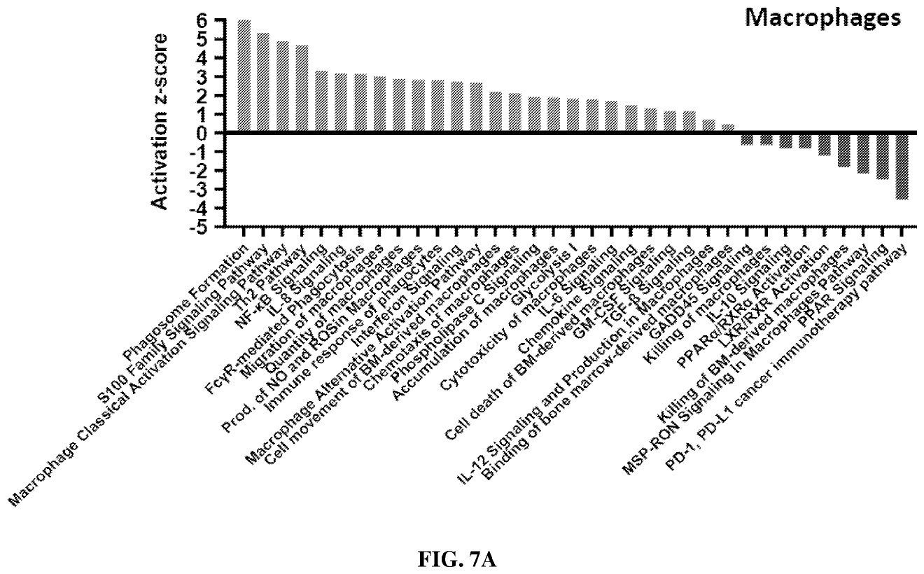

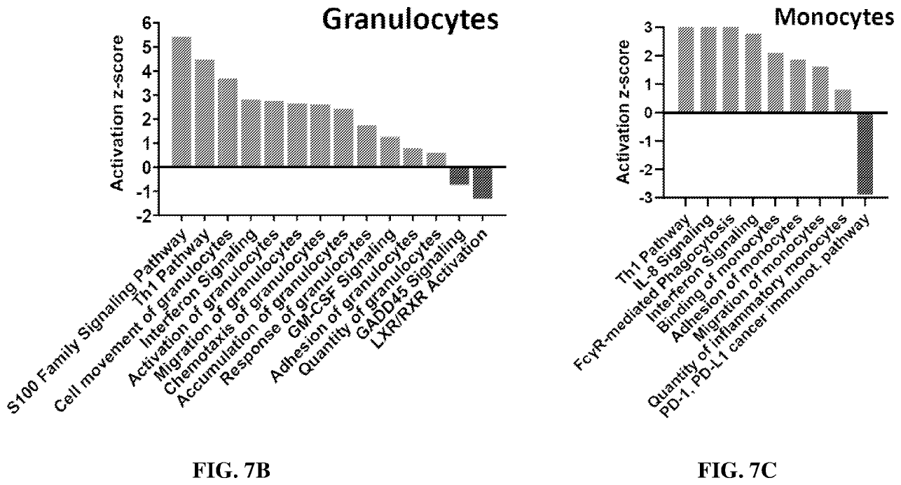

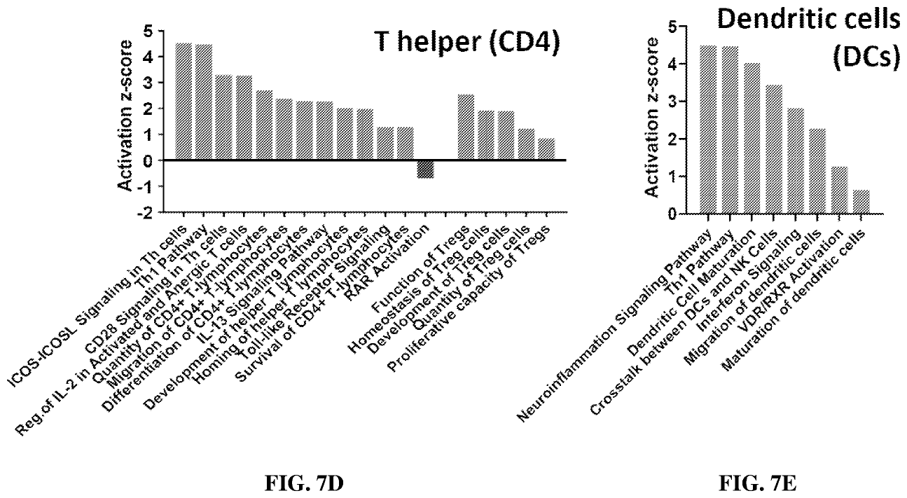

Importantly, the gene enrichment analysis investigated by IPA pointed out that the treatment of LGG animals with GEMys-IL2 triggered the upregulation of signaling pathways and biological functions associated with trafficking and activation of cytotoxicity in CD8+ cells (FIG. 5A). Comparable results were obtained for NK cells (FIG. 5B) and DC cells. The recruitment of GEMys-IL2 at the TME was also associated with the reprogramming of immunosuppressive and M2-like macrophages to become more pro-inflammatory (FIG. 7A). The software also predicted the upregulation of pathways and biological functions correlated with the activation and recruitment of granulocytes, monocytes, and T helper cells (FIG. 7B-7D). In addition, the IPA analysis on the RNAseq signature did not produce any significant data in support of upregulation of pathways or biological functions involved in T cell inactivation, or exhaustion due to the treatment with GEMys-IL2. Taken together, these results proved that the transcriptome of immune cells in the tumor microenvironment of LGG mice can be significantly influenced by the recruitment of naïve myeloid cells engineered for the release of the pro-inflammatory payload in the TME (GEMys-IL2).

GEMys-IL2 Potentiate a Pro-Inflammatory Immune Cell Composition within the Glioma Microenvironment

Our determination that GEMys-IL2 infiltrate the TME and efficiently secrete IL2 to reprogram the gene expression of immune cells in a murine LGG model did not elucidate whether they could potentiate the trafficking and activation of cytotoxic T, and NK cells. To address this issue, we engrafted LGG animals with GEMys-IL2 and we first characterized the inflammation in the TME by cytokine array (FIG. 6E). As negative control, we investigated the inflammation in the TME of mice engrafted with vehicle (PBS). The analysis (FIG. 5C) pointed out the significant upregulation of cytokines in the TME involved in the trafficking of myeloid cells (CCL2, CCL22, CCL3, CXCL2), DCs (CCL2, CCL20, RBP4), NK (CCL2, CCL22, CXCL9, CXCL10) and T cells (CCL2, CCL22, CCL20, CXCL9, CXCL10, IGFBP-5, RBP4). Importantly, we measured a significant upregulation of cytokines involved in the activation of myeloid cells (CD14, M-CSF, IFNy), as well as in the activation of T cells (TIM-1, IL2, IFNy, SAP), cytotoxic T cells (IL2, CD26, CD160), and NK cells (IL2, SAP, CD160). In addition, the IL2 and IFNy upregulation in the glioma microenvironment in vivo and post-treatment were confirmed by ELISA (FIG. 6F).

One of the aims for this study was to demonstrate that the bone marrow-derived myeloid cells engineered for the release of Interleukin-2 (GEMys-IL2) within the TME were able to recruit and activate anti-glioma cytotoxic T lymphocytes and arrest the tumor progression. Therefore, we investigated the composition of the immune cells isolated from the glioma microenvironment 3 days post-treatment with GEMys-IL2 by mass cytometry (FIG. 5F). In support of our hypothesis (FIG. 3F), the treatment of RCAS/t-va animals with GEMys-IL2 stimulated the trafficking of T lymphocytes (CD45+CD3+) to the TME. Interestingly, we also found more infiltration of activated cytotoxic T cells (CD45+CD3+CD8+CD25+) and activated NK cells (CD45+CD3−CD19−CD11b−nkp46+) (FIG. 5F). The results concerning the infiltration and activation of regulatory T lymphocytes (Tregs) after treatment with GEMys-IL2 in vivo were controversial. Interleukin-2 promotes proliferation and differentiation of cytotoxic T cells, but also differentiation and homeostasis of regulatory Tregs. Bensinger et al., The Journal of Immunology 172, 5287-5296 (2004). IPA analysis predicted the upregulation post-treatment of genes associated proliferation and trafficking of Tregs (FIG. 7D), but the analysis of the tumor infiltrating T cells by mass cytometry did not show a statistically relevant increased infiltration of Tregs (CD45+CD4+CD25hiFoxP3+) (FIG. 6D). In addition, the treatment did not induce any significant change in expression of FoxP3 in the tumor infiltrating immune cells in vivo (FIG. 4E). FoxP3 is the specific transcription factor for immunosuppressive Tregs. Taken together, these results confirmed that the treatment with GEMys-IL2 of LGG in vivo is associated with a pro-inflammatory and less immunosuppressive TME, with more recruitment of activated cytotoxic T lymphocytes and NK cells, thus suggesting a therapeutic role for this innovative cell-mediated immunotherapy that warrants further investigation.

### GEMys-IL2 Homing to the Glioma TME Delays Malignant Progression

To investigate whether the treatment of LGGs in vivo had an impact on the tumor burden, we employed the RCAS/t-va murine model. We engrafted GEMys-IL2 in randomized LGG animals at week 4 post-gliomagenesis, as previously described (Hambardzumyan et al., Transl Oncol 2, 89-95 (2009)), to understand whether the reduced tumor burden was associated with an improved survival in vivo. We conducted a survival study and followed the tumor progression of these animals weekly by IVIS. As shown in the Kaplan-Meier survival curve, a single injection of eight million syngeneic GEMys-IL2 cells was sufficient to induce a significantly prolonged survival in RCAS/t-va LGGs animals compared to animals injected with vehicle (PBS, p=0.0006) (FIG. 5G). Longitudinal IVIS monitoring demonstrated that, after an initial and remarkable reduction in tumor progression after seven days in animals treated with GEMys-IL2 compared with control animals (PBS), tumor relapse began two weeks post-treatment (FIG. 5H). Indeed, circulating IL2 levels in serum at the endpoints were similar in animals treated with a single dose of GEMys-IL2 or vehicles (FIG. 6G). Moreover, all the animals at the endpoints had unequivocal transformation to HGG (FIG. 6H); therefore, suggesting the effect of a single GEMys-IL2 treatment does not last longer than 7-14 days (FIG. 5G,H; FIG. 6G,H). Although, it delayed malignant progression significantly.

### DISCUSSION

Here we report for the first time the successful use of a novel cell-mediated innate immunotherapy in a pre-clinical LGG to HGG progression model to delay glioma malignant progression. This novel immunotherapeutic approach consisted of the treatment of LGG bearing animals with syngeneic bone marrow-derived myeloid cells, engineered for the stable release of Interleukin-2 (GEMys-IL2) into the glioma microenvironment. In the past, myeloid cells were considered just therapeutic targets in cancer, but new studies are revisiting their activity as potential therapeutic agents given their plasticity. Canella et al., Cancer Gene Ther., 30(7):964-972 (2023). To that end, two recent studies demonstrated the in vivo effect of primary derived and engineered myeloid cells for solid tumors. Specifically, a study published in 2020 evaluated the efficacy of treating ovarian cancer in vivo with human macrophages engineered to bind Her2 (anti-HER2 CAR-Ms). The second study from 2021 demonstrated the therapeutic efficacy of treating pre-metastatic lung cancer in a murine model with bone marrow-derived myeloid cells engineered for the release of the pro-inflammatory cytokine IL12. Kaczanowkska et al., Cell 184, 2033-2052 e2021 (2021). The GEMys we generated in vitro were comprised of several types of mature and naïve myeloid cells. Upon characterization, these myeloid cells were comprised of neutrophils, monocytes, and macrophages, but it remains to be determined whether the anti-cancer and pro-inflammatory activity in vitro and in vivo was mediated by a defined myeloid cell population or by diverse transduced myeloid cells. In addition, the treatment was delivered intravenously and well tolerated in an immunocompetent syngeneic LGG murine model. Recently, we proved that systemically injected bone marrow cells in glioma animals were able to pass the BBB and be recruited into the TME. Rajappa et al., Clin Cancer Res 23, 3109-3119 (2017). In a similar fashion, this study demonstrates that GEMys injected intravenously into immunocompetent glioma-bearing mice were recruited to and infiltrated the local tumor microenvironment.

Once in the TME the GEMys-released IL2 and our data suggests the pro-inflammatory nature of these naïve myeloid cells and the constant secretion of IL2 potentiates a pro-inflammatory TME and activation of the infiltrating immune cells. Four days post-treatment with GEMys-IL2, we observed increased infiltration of immune cells in the LGG TME in addition to increased trafficking and activation of cytotoxic T and NK cells. This treatment effect was associated with pro-inflammatory reprogramming of the infiltrating immune cells in vivo as demonstrated by the upregulation of pro-inflammatory cytokines for T, NK, and myeloid cell activation. This observation aligned with a pro-inflammatory and activated transcriptomic status further supported by downregulation of the immunosuppression marker FoxP3 and lower or no difference in the gene expression of exhaustion markers Lag3 and Tim-3. The immune cells were recruited to the TME and stimulated by the GEMys-IL2, inducing upregulation of IFNγ and STAT1 expression, and together with IL2 are all pro-inflammatory and pro-activating molecules. Of note, we did not measure any significant increased infiltration of the immunosuppressive regulatory T lymphocytes (Tregs).

In addition, evaluating the number of GEMys infiltrating the TME five days post-treatment with respect to the number of cells systemically injected is challenging due to cell state plasticity within the TME. However, one single dose of GEMys in LGG mice was sufficient to delay glioma malignant progression and to significantly improve overall survival, but it was not curative. Upon survival study endpoint, we observed neither GFP positive myeloid cells in the TME of animals treated with GEMys-IL2 compared with control animals nor an increased amount of IL2 in the peripheral blood. Our results suggest that the GEMys effect in the TME is transient and appears to have therapeutic efficacy of approximately seven to fourteen days in the treated animals. However, it remains unclear if the GEMys disappear by physiological turnover, are reprogrammed to be immunosuppressive after recruitment into the TME, or whether the prolonged stimulation of T cells with IL2 is causing exhaustion or inactivation. Future studies will address these fundamentally important questions. In addition, functional evaluation of the spatial distribution of other innate and adaptive immune cells will be critical.

In previous investigations using a similar glioma immunocompetent model, tumors were locally treated by injection of mature macrophages engineered for the release of IL12 into the tumor bed. However, the effect on the transcriptome was reached exclusively by intratumoral injection and no survival data on the model was reported (Brempelis et al., J Immunother Cancer, 8(2):e001356 (2020)), hence the clinical relevance of the findings appear to be unclear. In fact, although intratumoral delivery of drugs may be feasible in more accessible solid tumors, the translation of such therapy to the clinic is still challenging for brain tumors. In the past, the treatment of glioma patients with intratumoral or systemic injections of recombinant interleukin-2 (rIL2) was evaluated, but with scarce success. After initial encouraging therapeutic results on controlling tumor progression, the high dosage required to reach a therapeutic effect and the consequent development of severe toxicity was noted in several patients. In mice, the intracranial injection of rIL2 caused the disruption of the blood brain barrier (BBB) and the consequent development of cerebral edema. In patients, the intracranial injection of rIL2 was associated with toxicity and development of IL2-induced capillary leak syndrome, edema around the tumor, thrombocytopenia, cardiac arrhythmias, hepatic dysfunction, and fever. Therefore, the development of severe toxicity has been a challenge for the direct delivery of rIL2 into the tumor bed of patients with glioma so far.

This study is the first to demonstrate the pre-clinical therapeutic activity of bone marrow-derived naïve and pro-inflammatory myeloid cells, engineered to express and secrete IL2 in the TME, which delayed malignant progression of low-grade gliomas and prolong OS. In addition, the systemic GEMys treatment approach is a novel concept that leverages tumor homing mechanisms given the crosstalk between tumor and host innate immunity. Due to a well-tolerated treatment course in our pre-clinical models with GEMys and the positive effect on the recruitment of effector T cells, we believe the data suggests that treatment may be more potent if the animals are treated with multiple administrations of GEMys or in combination with checkpoint inhibitors. These approaches may augment or help maintain cytotoxic T cell trafficking and activation and further studies are required given the limitations in first-line immunotherapy regimens observed in clinical care currently.

In conclusion, this preclinical study highlights a potential novel tool in developing a next-generation innate systemic immunotherapy.

### Methods

Generation of genetically engineered myeloid cells for the stable expression of IL2. The generation in vitro of control GEMys (empty vector, EV) or GEMys for the stable expression and release of IL2 (GEMys-IL2) was performed as previously described. Kaczanowska et al., Cell 184, 2033-2052 e2021 (2021). Briefly, bone marrow cells were isolated from mice not bearing tumors (NTV-a;Ink4a^{+/-} Arf^{+/-};PTEN^{+/fl};LSL-Luc). The hematopoietic progenitors were isolated by magnetic negative selection (Mouse Hematopoietic Progenitor Cell Isolation Kit, StemCell Technologies) and cultivated in SFEM II media supplemented with 50 ng/ml of murine IL6, SCF, and FLT3/Flk-2 ligand, (StemCell Technologies) and 1% penicillin/streptomycin to drive the differentiation to mature myeloid cells. After 2 hours, cells were infected with lentiviral particles (MOI=100) in the presence of Polybrene 10 μg/ml (Sigma-Aldrich). The lentiviral particles were generated by co-transfection in Lenti-X™ 293T cells (Takarabio) of lentiviral packaging kit (Origene) and plasmids for the expression of IL2-GFP+ or the empty vector-GFP+, according to the manufacturer's protocol (Origene). The lentivirus concentration was quantified by Lenti ELISA Kit (Origene). After 16 h, the cells were scraped, washed, and seeded in fresh media. Three days post-infection, the cells were evaluated for the expression of IL2 by quantitative real-time PCR (RT-qPCR) and for the release of IL2 by ELISA (R&D Systems). Infected myeloid cells were selected by the addition of puromycin 1 μg/ml for 2 weeks and both the GFP and IL2 expression were monitored weekly by flow cytometry (LSR Fortessa, BD Biosciences), fluorescent microscopy (EVOS, AMG), RT-qPCR (Applied Biosystems), and ELISA (R&D Systems).

### Mouse Model

NTV-a;Ink4a^{+/-} Arf^{+/-};PTEN^{+/fl};LSL-Luc and the RCAS system were adopted to undergo gliomagenesis in vivo. Holland et al., Genes Dev 12, 3675-3685 (1998). DF-1 chicken fibroblasts transfected with RCAS-PDGFb and DF-1 cells transfected with RCAS-Cre were maintained in DMEM with 10% fetal bovine serum (Gibco). Heterozygous NTV-a; Ink4a^{+/-} Arf^{+/-};PTEN^{+/fl};LSL-Luc were generated by crossing NTV-a (WT) mice with NTV-a; Ink4a^{-/-} Arf^{-/-};PTEN^{fl/fl};LSL-Luc mice. 0-2 days after birth, heterozygous pups were injected with 1 μL DPBS (Gibco) containing the two fibroblast cell lines (5×10⁵ cells/kind). Injections were executed in the right hemisphere of the brain parenchyma, 3 mm lateral to the bregma at a depth of 1 mm. When mice were 4 weeks old, D-luciferin (Perkin Elmer) was administered by IP injection, and tumor burden was monitored by bioluminescence imaging (IVIS Spectrum, Perkin Elmer).

Evaluation of the treatment in the TME. Gliomagenesis was induced by injection of DF1-Cre and DF1-PDGF cells in NTV-a; Ink4a^{+/-} Arf^{+/-};PTEN^{+/fl};LSL-Luc pups at 0 to 2 days of age, as already described. At 4 weeks old, tumor burden was evaluated by In Vivo Imaging System (IVIS) followed by mice randomization and separation into 3 treatment groups. On day 28, mice were retro-orbitally injected with 80 μl of DPBS (vehicle) or with 8 million of GEMys-EV or GEMys-IL2. Mice were monitored twice a day and euthanized at day 5 or day 7 post-treatment followed by the isolation of the brain and other tissues for processing for further investigation.

Survival studies. Gliomagenesis was induced by injection of DF1-Cre and DF1-PDGF cells in NTV-a; Ink4a^{+/-} Arf^{+/-};PTEN^{+/fl};LSL-Luc pups at 0 to 2 days of age, as previously described. At 4 weeks of age, tumor burden was evaluated by IVIS followed by mice randomization and separation in 3 treatment groups. On day 28, mice were retro-orbitally injected with 80 μl of DPBS (vehicle), 8 million of GEMys-EV, or 8 million of GEMys-IL2. Mice were monitored twice a day and euthanized at the beginning of decline, with symptoms such as lethargy, weight loss, macrocephaly, seizure, hyperactivity, or abnormal behavior.

Tissue harvesting. Bone Marrow: Femurs and tibias were dissected in sterility from NTV-a; Ink4a^{+/-} Arf^{+/-};PTEN^{+/fl};LSL-Luc mice not bearing tumors at 4 weeks of age. Bone marrow was extracted by centrifugation at 10,000×g for 30 seconds. Spleen: Spleens were dissected from NTV-a; Ink4a^{+/-} Arf^{+/-};PTEN^{+/fl};LSL-Luc mice without tumors at 4 weeks of age. Peripheral Blood: At endpoint, mice were anesthetized with an isoflurane vaporizer (Kent Scientific) and cardiac puncture was performed to collect 400 μl of peripheral blood. Blood was transferred into microtainer EDTA blood collection tubes (BD), and animals were immediately euthanized. Blood samples were centrifuged at 2,000×g for 10 minutes at +4 °C, serum was collected and immediately analyzed by ELISA or stored at −80 °C. Brain: Whole brains were dissected from mice at endpoint and fixed in 10% buffered formalin phosphate for 24 hours for histology. The tumor area was also extracted from the whole right hemisphere with a scalpel and digested to investigate the inflammatory status (by cytokine array). Alternatively, the tumor area was dissociated to isolate immune cells of the glioma microenvironment to assess cellular composition (by CyTOF and flow cytometry) and perform analysis of the transcriptome (by RT-qPCR and Nanostring).

### Isolation of Immune Cells Infiltrating the Glioma Microenvironment

Animals were euthanized 5 days post treatment, brains were removed, and the area around the tumor was isolated from the rest of the right hemisphere. The brain tissue was mechanically dissociated on a 70 μm cell strainer and collected in DPBS supplemented with the 10% of FBS (Corning). Cells were washed (DPBS+10% FBS), centrifuged at 300 g for 6 minutes at +4 °C, and resuspended with RBC buffer (Biolegend) on ice for 5 minutes to lyse the red blood cells. RBC solution was quenched with 5×DPBS+10% FBS and cells isolated by centrifugation. The stratification with 25% Percoll (GE Healthcare) was performed to deplete the myelin layer at 500 g for 20 minutes at 18 °C in absence of break. The pellet was washed in DPBS+ 10% FBS and stained for flow cytometry or processed for RNA isolation and analysis of the transcriptome.

Single Cell RNA Sequencing (scRNAseq)

Tumor or normal tissue was excised from the brain's right hemisphere at the injection site. The tissue was then dissected into smaller pieces, digested with 2 mg of Papain (Brainbits) for 20 minutes at 37 °C, and filtered through a 70 μm cell strainer. CD45+ cells were isolated by magnetic positive selection (Miltenyi). Sequencing was performed by the Genomic core facility of the Nationwide Children's Hospital of Columbus, Ohio. Samples were run on a Chromium controller (10X Genomics), using Next GEM Single cell 3′ Reagent kit 3.1. Results were mapped to the mm10 mouse genome reference using CellRangerv3.0.2 (10XGenomics). Seurat v.4 and ShinyCell packages were utilized to evaluate gene expression analysis in R., and scRNAseq data were deposited to GEO (GSE221440). The database generated for the Ingenuity Pathway Analysis (IPA, Qiagen) is publicly available.

### Total (Bulk) RNA Sequencing.

Total RNA was extracted from the immune cells infiltrating the glioma microenvironment after three days post treatment of LGG RCAS/t-va mice with vehicle (PBS) or 8×10⁶ GEMys-IL2 (n=3), using RNeasy micro kit (Qiagen). Quality was assessed using Agilent Bioanalyzer RNA chips, and quantification via Qubit RNA High Sensitivity assay. Libraries were generated with NEBNext Ultra II Directional RNA prep (Biolabs) and sequenced with Illumina NovaSeq6000 (Illumina). Low quality reads (q<10) and adaptor sequences were eliminated from raw reads using bbduk version 37.64; each sample was aligned to the GRCm38.p6 assembly of the Mus musculus reference from NCBI, using version 2.6.0c of the RNA-Seq aligner STAR; features were identified from the GFF file that came with the assembly from Gencode (V28), and coverage counts were calculated using featureCounts. The library preparation, RNA sequencing, data alignment and normalization were performed by the Genomic Core Facility of the Nationwide Children's Hospital (Columbus, Ohio). Sequencing data were deposited to NCBI GEO (GSE233646). P-values were calculated using the Student's t-test. Genes with a p-values<0.05 were retained for the downstream analyses. Clustering analysis was performed with Cluster 3.0, and the heatmap was generated with Java Treeview. Ingenuity Pathway Analysis (IPA) was used to predict disease-related functions, upstream regulators, networks, and pathways affected by the tumor progression or by the treatment in vivo.

### Quantitative Real Time PCR (RT-qPCR)

Total RNA from primary isolated murine cytotoxic T cells and myeloid cells was extracted using RNeasy mini kit (Qiagen), and total RNA from primary immune cells isolated in the glioma microenvironment was extracted using RNeasy micro kit (Qiagen). RNA quantity and quality were assessed by Nanodrop (Thermo Fisher), and cDNA was synthetized using High-Capacity cDNA Reverse Transcription Kit (Applied Biosystems) in accordance with the manufacturer's protocol. SYBR Green RT-qPCR experiments were executed following the manufacturer's protocols (Applied Biosystems) using PrimeTime qPCR primer assays (IDT), and in a StepOnePlus system (Applied Biosystems).

### Mass Cytometry (CyTOF) and Flow Cytometry

GEMys Composition. To determine the composition of GEMys in vitro, a large twenty-six parameter mass cytometry (CyTOF) panel consisting of both innate and adaptive immune cell markers was applied to both GEMys-EV and GEMys-IL2 in triplicate to ensure consistency in the generation of GEMys. In brief, isolated immune cells were washed twice with Maxpar Cell Staining buffer (Fluidigm), incubated in Fc-blocking solution for 10 minutes, stained with 100 μL of a cocktail of metal-conjugated surface antibodies and incubated for 30 minutes at 4 °C with vortexing. Stained cells were washed twice with PBS, fixed with 1.4× Proteomic stabilizer PROT1 buffer (Smart Tube Inc.) for 10 minutes at room temperature, and stored at −80 °C. Samples were acquired on Helios mass cytometer (Fluidigm) and analyzed with OMIQ (Dotmatics) to produce UMAP and FLOWSOM clustering. Manual gating was used to confirm cluster phenotypes in accordance with those reported. Becher et al., Nat Immunol 15, 1181-1189 (2014).

Further, smaller titrated myeloid flow cytometry panels were designed with FMO controls to validate the CyTOF data and assess c-Kit expression. Cells were washed in PBS, stained with ZOMBIE NIR (Biolegend 423105) for 18 minutes, resuspended in Fc-blocking solution (BD Biosciences 553142) for 10 minutes, and incubated for 45 minutes with either CD45-PE (Biolegend 103106), CD11b-BV421 (Biolegend 101235), F4/80-APC (Biolegend 123116), Gr1-BV650 (Biolegend 108441), CD115-PeCy7 (Biolegend 135523), CD11c-PerCP/Cy5.5 (Biolegend 117327), and IA/IE-BV786 (Biolegend 107645), or CD45-PE (Biolegend 103106), CD11b-BV421 (Biolegend 101235), CD44-BV510 (Biolegend 103043), c-kit-APC (Biolegend 105811), and FceRal-PECy7 (Biolegend 134317). All the incubations were performed on ice and in the dark. After 2 washes with PBS, samples were fixed in 2% PFA, acquired using LSR Fortessa (BD Biosciences), and analyzed with FlowJo-v10. Fluorescence minus one (FMO) controls were used to determine positive gates for c-kit, IA/IE, CD11c, CD115, and Gr-1.

CD8+ T cells co-culture with GEMys. Spleens were harvested from 4-week-old NTV-a/Ink4a^{+/-} Arf^{+/-}/PTEN^{fl}/LSL-Luc mice without tumors and immediately mechanically dissociated on a 70 μm cell strainer (Thermo Scientific). Cells were washed in PBS and processed for CD8+ selection (StemCell Technologies). Next, T cells were co-cultured in 24 well plates with GEMys-EV or GEMys-IL2 with a T-cell:GEMys ratio of 1:3 for 1 to 4 days in RPMI media supplemented with 10% FBS (Gibco). To profile the transcriptome, CD8+ cells were co-cultured with GEMys-EV or GEMys-IL2 for 24 hrs, followed by CD8+ T cells magnetic negative selection (StemCell Technologies), total RNA extraction, and real-time PCR. Mass cytometry and flow cytometry were used to evaluate the protein expression. For extracellular staining and acquisition, a protocol outlined by Patel et al. was adapted for murine samples. Cytotechnology 70, 1-11 (2018). To evaluate the activation status of the cytotoxic T lymphocytes (CTL) by mass cytometry, cells were harvested after 48 hours of co-culture with the GEMys, washed in PBS, stained as previously described, acquired on Helios mass cytometer (Fluidigm), and analyzed with OMIQ (Dotmatics) to produce UMAPs. To evaluate the activation status of the cytotoxic T lymphocytes (CTL) from the GEMys co-culture by flow cytometry, cells were harvested after 4 days, washed in PBS, stained with a panel of titrated antibodies including ZOMBIE NIR (Biolegend 423105), CD45-PE (Biolegend 103106), CD3-PECy7 (Biolegend 100319), CD8-BV786 (Biolegend 100749), and CD69-BV421 (Biolegend 104527). CTLs were gated as viable CD45+ CD3+ CD8+ and their activation status was assessed by CD69 with florescence minus one (FMO) control for each activation marker. As positive controls for activation, CTLs were stimulated for 24 and 48 hr with anti-CD3/28 Dynabeads (Thermofischer 11456D) as described by Patel et al. Stained cells were acquired on a LSR Fortessa (BD Biosciences) and analyzed using FlowJo software.

Immune profile of the Glioma Microenvironment. Immune cells were isolated as described above and analyzed by CyTOF and Flow Cytometry. Mass cytometry (CyTOF): Cells were stained as already described, followed by an intracellular foxp3 stain. Fixed cells were resuspended in methanol, incubated at −20 °C for 15 min, washed 3× with CSM, incubated with a foxp3 cocktail for 50 min on shaker, washed with PBS, resuspended in PBS+1.5% PFA+1:4000 Intercalator for 30 min, and washed 1×CSM and 2× water before acquiring on the Helios mass cytometer (Fluidigm). Data were analyzed with OMIQ (Dotmatics) to produce UMAPs. Flow Cytometry: cells were stained with ZOMBIE NIR (Biolegend 423105), and CD45-PE (Biolegend 103106). Since both the EV and IL-2 GEMys express GFP, fluorescence was measured in the FITC channel.

Immunofluorescence. Whole brains were dissected from mice at endpoint and fixed in 10% buffered formalin phosphate for 24 hrs. 5 m paraffin-embedded brain tissue sections were obtained from mice treated with vehicle, GEMys-EV, or GEMys-IL2, and euthanized 5 days post treatment. After deparaffinization and hydration, antigen retrieval was performed in citrate pH 6.0 buffer on a high-pressure setting. The sections were blocked with TBST with 10% normal goat serum for one hour at room temperature. Sections were treated with nuclear stain Hoescht (1:5000, Life Technologies) for 10 minutes at RT and mounted using Prolong gold anti-fade mounting media. 20× images were acquired with the ImageXpress Micro Confocal High-Content Imaging System (IXMC, Molecular Devices). To measure the infiltration of GFP positive cells in the TME, the percentage of positive cells to GFP, and Hoescht colocalization were calculated by the multiwavelength scoring module of the MetaXpress software (Molecular Devices).

Cytokine array. Immune cells infiltrating the glioma microenvironment in the RCAS murine model were collected from the tumoral mass in the right hemisphere of brains from animals at week 3 (low-grade glioma), or at week 7 (high-grade glioma). Tumor progression was validated by histological evaluation of 5 m paraffin-embedded brain tissue sections stained by H&E. Total protein lysates were generated with RIPA buffer supplemented with HALT proteases inhibitor cocktail (Thermo Scientific), and the total protein concentration was quantified by BCA (Thermo Scientific), per manufacturer's protocol recommendations. The cytokine profile in FIG. 1G was investigated by Mouse Cytokine 44-plex Discovery Assay (Discovery Assay), and in FIG. 5C by mouse XL cytokine array in accordance with the manufacturer's instruction (R&D Systems).

Enzyme-Linked immunosorbent Assay (ELISA). To perform the quantification of the circulating murine IL2 in the peripheral blood, or of the released IL2 in the culture media, we performed ELISA as described by the manufacturer (R&D Systems). All incubations were conducted at room temperature unless otherwise noted. Briefly, the plate was coated overnight with 100 μL of primary antibody and blocked in 1% BSA for 1 h. 100 μL samples or standards were seeded in triplicate and incubated for 2 hrs. The plate was washed, and biotinylated anti-IL2 was added to each well and incubated for 2 hrs. 100 μL Streptavidin-HRP was added and allowed to stand for 20 min. In the dark, 100 μL of substrate solution was added to each well and incubated for 15 min. 50 μL of stop solution was added and absorbance at 450 nm and 570 nm (wavelength correction) was read on a Synergy 2 microplate reader (Biotek).

Quantification and statistical analysis. All results were generated by multiple independent observations and expressed as the mean±standard deviation (SD). Overall survival was defined as the time from the injection of cells to generate gliomagenesis to the time of the endpoint and measured by Kaplan-Meier curve. Survival analyses were performed using Log-rank (Mantel-Cox) test. All reported p-values were calculated by unpaired two-tailed t-test and analyses were performed using GraphPad (Prism). Experiments generated by at least three independent experiments with a p-value<0.05 were considered significant.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

What is claimed is:

1. A method of treating or preventing central nervous system cancer in a subject in need thereof, comprising administering to the subject a therapeutically effective amount of myeloid cells modified to express interleukin-2.

2. The method of claim 1, wherein the central nervous system cancer is a glioma.

3. The method of claim 2, wherein the glioma is a low-grade glioma.

4. The method of claim 3, wherein treatment prevents progression to high-grade glioma.

5. The method of claim 1, wherein the method delays malignant progression.

6. The method of claim 1, wherein the myloid cells are bone marrow-derived.

7. The method of claim 1, wherein the myeloid cells comprise monocytes, eosinophils, mast cells, and myloid precursor cells.

8. The method of claim 1, wherein central nervous system cancer is treated.

9. The method of claim 1, wherein central nervous system cancer is prevented.

10. The method of claim 1, wherein the subject is further treated with chemotherapy or radiotherapy.

11. A population of genetically engineered myeloid cells (GEMys) comprising bone marrow derived myeloid cells that have been genetically modified to express interleukin-2.

12. The population of claim 11, wherein the myeloid cells are genetically engineered using a lentivirus vector.

13. The population of claim 11, wherein the population comprises monocytes, eosinophils, mast cells, and myloid precursor cells.

14. The population of claim 13, wherein population comprises at least 15% monocytes, at least 15% eosinophils, at least 15% mast cells, and at least 15% myloid precursor cells.
